(19) United States
(12) Patent Application Publication
(10) Pub. No.: US 2003/0232417 A1
(43) Pub. Date: Dec. 18, 2003

(54) Process

(76) Inventors: Andrew John Morgan, Vedbaek (DK); Shukun Yu, Malmoe (SE)

Correspondence Address: FROMMER LAWRENCE & HAUG, 745 FIFTH AVENUE- 10TH FL., NEW YORK, NY, 10151, US

(21) Appl. No.: 10/283,963

(22) Filed: Oct. 30, 2002

Related U.S. Application Data
(60) Provisional application No. 60/343,316, filed on Dec. 21, 2001.
(60) Provisional application No. 60/343,447, filed on Dec. 21, 2001.
(60) Provisional application No. 60/343,485, filed on Dec. 21, 2001.
(60) Provisional application No. 60/343,368, filed on Dec. 21, 2001.
(60) Provisional application No. 60/343,313, filed on Dec. 21, 2001.

(30) Foreign Application Priority Data
Oct. 31, 2001 (GB) 0126162.7

(51) Int. Cl. C12P 17/06

(52) U.S. Cl. 435/125

(57) ABSTRACT

The present invention relates to a process for preparing ascopyrone P, or a derivative thereof, said process comprising the steps of:

(I) converting a starch-type substrate to 1,5-anhydro-D-fructose with α-1,4-glucan lyase at a pH of from about 3.8 to 7.0;

(II) treating said 1,5-anhydro-D-fructose with 1,5-anhydro-D-fructose dehydratase and/or pyranosone dehydratase and optionally ascopyrone P synthase at a pH of from about 5.0 to about 7.5.

### CROSS REFERENCE TO RELATED APPLICATIONS

This application claims priority to provisional application serial No. 60/343,316, filed Dec. 21, 2001 entitled “Process,” and U.K. application no. 0126162.7 filed Oct. 31, 2001; both of which are incorporated herein by reference, together with any documents therein cited and any documents cited or referenced in therein cited documents. Reference is made to U.S. Provisional Patent Applications Serial Nos.: 60/343,447, filed Dec. 21, 2001, entitled “1,5-Anhydro-D-Fructose Dehydratase”; 60/343,485, filed Dec. 21, 2001, entitled “Sequences”; 60/343,368, filed Dec. 21, 2001, entitled “Use” and 60/343,313, filed Dec. 21, 2001, entitled “Ascopyrone P Synthase”; incorporated herein by reference, together with any documents therein cited and any documents cited or referenced in therein cited documents. Reference is also made to the U.S. Utility Patent Applications based on the four referenced U.S. Provisional Patent Applications which are field concurrently herewith as Attorney reference Nos.: 674509-2042.1, 674509-2041.1, 674509-2039.1 and 674509-2040.1. All documents cited herein and all documents cited or referenced in herein cited documents are hereby incorporated herein by reference.

### FIELD OF THE INVENTION

The present invention relates to methods for the preparation of ascopyrone P and derivatives thereof.

### TECHNICAL BACKGROUND AND PRIOR ART

Starch and glycogen, the carbon storage polymers in plants, bacteria, fungi and animals are usually degraded to form glucose, and oligomers thereof, and glucose 1-phosphate upon their need. These processes are catalyzed by hydrolases and phosphorylases, respectively. More recently, an alternative starch/glycogen degradation pathway has been elucidated in fungi and red algae. Two enzymes have been identified in this novel catabolic pathway, which is also known as the anhydrofructose pathway. α-1,4-Glucan lyase (EC 4.2.2.13) is the first enzyme that catalyzes the breakdown of glycogen to form 1,5-anhydro-D-fructose (AF) [Yu, S.; K. Bojsen, B. Svensson, and J. Marcussen, Biochim. Biophys. Acta. 1433(1-2) (1999): 1-15]. The AF is then converted by AF-dehydratase (AFDH) to form a precursor molecule, which is subsequently converted to a range of secondary metabolites, such as ascopyrone P in the case of Anthracobia melaloma.

Ascopyrone P or APP (1,5-anhydro-4-deoxy-D-glycero-hex-1-en-3-ulose) was first prepared from the pyrolysis of amylopectin, amylose and cellulose in a yield under 3% [Shafizadeh, F., Fumeaux R. H., Stevenson, T. T., and Cochran, T. G., Carbohydr. Res. 67(1978): 433-447]. It has since been further characterised and its crystal structure has been reported [Stevenson, T. T., Stenkmap, R. E., Jensen, L. H., Cochran, T. T., Shafizadeh, F., and Fumeaux R. H., Carbohydr. Res. 90(1981): 319-325] APP has been isolated from the fungi of the order of Pezizales, such as Anthracobia melaloma, Plicaria anthracina, P. ieiocarpa, and Peziza petersi (Baute et al. 1993). APP has been found to be a good antioxidant, antibrowning agent and antimicrobial agent [WO 00/56838 filed Mar. 16, 2000, paragraphing priority from GB9906457.8, filed Mar. 19, 1999; WO 02/26060 filed Sep. 27, 2001, paragraphing priority from GB023686.9 and GB0023687.7, both filed Sep. 27, 2000].

However, the production of APP using the pyrolysis method is of little practical value because of the low yield [Shafiadeh et al., 1978]. The method of Baute et al [M.-A. Baute, G. Deffieux, J. Vercauteren, R. Baute, and A. Badoc, Phytochemistry, 33 (1993): 41-45] involved the use of a cell-free extract as the enzyme source. Again, the yield was low and the method is only of use for milligram scale preparation of APP. Although APP can be synthesised chemically from glucose and anhydrofructose, the yield is low and multiple steps are needed [Andersen, S. M.; Jensen H. M. (2001); Ascopyrone P: Chemical Synthesis from D-Glucose, in press].

It has been presumed by Baute et al. [1993, ibid] that APP can be formed enzymatically from glycogen in certain species of fungi belonging to the order Pezizales, but to date, none of these enzymes have been isolated, purified or characterised.

The AF derivative microthecin (2-hydroxy-2-(hydroxymethyl)-2H-pyran-3(6H)-one)) was first produced by fermentation of the fungus Microthecium and found to be antifungal against a limited number of fungi tested [Naito et al., 1978, laid open Japanese Patent Application No. 53-30381, Publication No. 54-122796]. However, there was no disclosure of the enzymes involved. Baute et al. [M.-A. Baute, G. Deffieux, R. Baute, A. Badoc., J. Vercauteren, J.-M. Léger, and A. Neveu; Fungal enzymic acitivity degrading 1,4-α-glucans to echinosporin (5-epipentenomycin I) Phytochemistry, 30 (1991): 1419-1423] have proposed that microthecin can be formed from AF by an undefined conventional dehydratase. However, to date the supposed dehydratase has not been purified or characterized.

The present invention seeks to provide an improved process for producing ascopyrone P and derivatives thereof that alleviates some of the problems associated with prior art processes, such as low yield and multi-step synthetic preparations.

### SUMMARY OF THE INVENTION

In a broad aspect the invention relates to an improved process for producing APP and derivatives thereof.

The applicant has established that three enzymes are needed for the conversion of glycogen, starch or dextrins to APP, namely, α-1,4-glucan lyase, AFDH and APS. The products of these three enzymes are AF, APP precursor (APM) and APP respectively.

More specifically, the invention discloses a process for producing ascopyrone P, or derivatives thereof, using α-1,4-glucan lyase, ascopyrone P synthase, and either 1,5-anhydro-D-fructose dehydratase or pyranosone dehydratase.

Aspects of the present invention are presented in the paragraphs and in the following commentary.

For ease of reference, these and further aspects of the present invention are now discussed under appropriate section headings. However, the teachings under each section are not necessarily limited to each particular section.

### DETAILED DISCLOSURE OF INVENTION

In a first aspect, the invention relates to a process for preparing ascopyrone P, or a derivative thereof, said process comprising the steps of:

(I) converting a starch-type substrate to 1,5-anhydro-D-fructose with α-1,4-glucan lyase at a pH of from about 3.8 to 7.0;

(II) treating said 1,5-anhydro-D-fructose with 1,5-anhydro-D-fructose dehydratase and/or pyranosone dehydratase and optionally ascopyrone P synthase at a pH of from about 5.0 to about 7.5.

In a preferred embodiment, steps (I) and (II) are carried out in a one-pot process by forming a reaction mixture comprising a starch-type substrate, α-1,4-glucan lyase, 1,5-anhydro-D-fructose dehydratase and/or pyranosone dehydratase, and optionally ascopyrone P synthase wherein the process is carried out at a pH of from about 5.0 to 7.5.

In a more preferred embodiment, steps (I) and (II) are carried out in a one-pot process at a pH of from about 5.0 to 7.0.

Another aspect of the invention relates to a process for producing ascopyrone P, or a derivative thereof, said process comprising the steps of:

(I) converting a starch-type substrate to 1,5-anhydro-D-fructose with α-1,4-glucan lyase;

(II) converting said 1,5-anhydro-D-fructose to ascopyrone P with ascopyrone P synthase and 1,5-anhydro-D-fructose dehydratase and/or pyranosone dehydratase;

and wherein the process is carried out at a pH of from about 5.0 to about 7.5.

Fungal glucan lyase may be purified and cloned in accordance with the methods described in Yu et al [Yu. S.; Christensen TMIE, Kragh K M, Bojsen K, Marcussen J, Biochim Biophys Acta, 1339: 311-320 (1997)].

Preferably, the concentration of starch-type substrate in the one-pot process is from about 2 to about 30% (w/v), preferably from about 10 to about 25% (w/v), preferably about 20% (w/v).

In one preferred embodiment, steps (I) and (II) are carried out sequentially.

Thus, in a particularly preferred embodiment, the process comprises:

(a) forming a reaction mixture comprising a starch-type substrate and α-1,4-glucan lyase; and

(b) adding 1,5-anhydro-D-fructose dehydratase and/or pyranosone dehydratase and optionally ascopyrone P synthase thereto.

Preferably, the process is carried out at a temperature from about 10° C. to about 75° C., preferably from about 22° C. to about 75° C., preferably from about 20° C. to about 40° C., preferably about 30° C.

In one preferred embodiment, the concentration of starch-type substrate when steps (I) and (II) are carried out sequentially is from about 2 to about 35% (w/v), preferably from about 10 to about 25% (w/v), preferably about 20%.

In another preferred embodiment, the process comprises:

(a) forming a first reaction mixture comprising a starch-type substrate and α-1,4-glucan lyase;

(b) isolating 1,5-anhydro-D-fructose obtained from said first reaction mixture;

(c) forming a second reaction mixture comprising 1,5-anhydro-D-fructose, 1,5-anhydro-D-fructose dehydratase and/or pyranosone dehydratase and optionally ascopyrone P synthase

In one particularly preferred embodiment, the 1,5-anhydro-D-fructose is isolated from the first reaction mixture by ultrafiltration.

Preferably, the concentration of 1,5-anhydro-D-fructose in said second reaction mixture is from about 0.4 to about 20% (w/v), preferably from about 5 to about 15% (w/v).

Preferably, steps (a), (b) and (c) are carried out at a temperature of from about 10° C. to about 45° C., preferably from about 22° C. to about 45° C., preferably from about 20° C. to about 40° C., preferably about 30° C.

In a preferred embodiment, the invention relates to a process for preparing ascopyrone P, said process comprising the steps of:

(I) converting a starch-type substrate to 1,5-anhydro-D-fructose with α-1,4-glucan lyase at a pH of from about 3.8 to 7.0;

(II) treating said 1,5-anhydro-D-fructose with 1,5-anhydro-D-fructose dehydratase or pyranosone dehydratase, and ascopyrone P synthase at a pH of from about 5.0 to about 7.5.

Preferably, steps (I) and (II) are carried out in a one-pot process by forming a reaction mixture comprising a starch-type substrate, α-1,4-glucan lyase, 1,5-anhydro-D-fructose dehydratase or pyranosone dehydratase, and ascopyrone P synthase, wherein said process is carried out at a pH of from about 5.0 to about 7.5.

In one preferred embodiment, said derivative of ascopyrone P is microthecin or ascopyrone M.

Thus, one preferred embodiment of the invention relates to a process for preparing microthecin, said process comprising the steps of:

(I) converting a starch-type substrate to 1,5-anhydro-D-fructose with α-1,4-glucan lyase at a pH of from about 3.8 to 7.0;

(II) converting said 1,5-anhydro-D-fructose to microthecin with pyranosone dehydratase and optionally 1,5-anhydro-D-fructose dehydratase at a pH of from about 5.0 to about 7.5.

Preferably, steps (I) and (II) are carried out in a one-pot process by forming a reaction mixture comprising a starch-type substrate, α-1,4-glucan lyase, pyranosone dehydratase and optionally 1,5-anhydro-D-fructose dehydratase, wherein the process is carried out at a pH of from about 5.0 to about 7.5.

Another preferred embodiment relates to a process for preparing ascopyrone M, said process comprising the steps of:

(I) converting a starch-type substrate to 1,5-anhydro-D-fructose with α-1,4-glucan lyase at a pH of from about 3.8 to 7.0;

(II) converting said 1,5-anhydro-D-fructose to ascopyrone M with pyranosone dehydratase or 1,5-anhydro-D-fructose dehydratase at a pH of from about 5.0 to about 7.5.

Preferably, steps (I) and (II) are carried out in a one-pot process by forming a reaction mixture comprising a starch-type substrate, α-1,4-glucan lyase, and pyranosone dehydratase or 1,5-anhydro-D-fructose dehydratase, wherein the process is carried out at a pH of from about 5.0 to about 7.5.

More preferably, for any of the above one-pot processes, the pH is from about 5.0 to about 7.0, even more preferably from about 6.0 to about 6.5.

As used herein, the term “starch-type substrate” includes, for example, glycogen, or an intermediate compound resulting from the hydrolysis of starch by amylase enzymes, such as a maltodextrin. Examples of starch-type substrates include starch, amylopectin, amylose and dextrin.

Preferably, the starch-type substrate is selected from starch, maltosaccharides, amylopectin, amylose and dextrin.

The process of the invention may also be used to prepare derivatives of ascopyrone P.

As used herein, the term “derivative of ascopyrone P” includes compounds having the general formula I

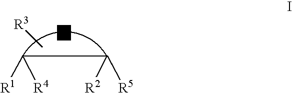

or a derivative thereof,

wherein

(a) R¹ and R² are independently selected from —OH, ═O, and OR′, wherein R′ is H or —COR″, and R″ is C₁₋₁₀alkyl;

R³ is a substituent comprising an —OH group;

R⁴ and R⁵ are each independently selected from a hydrocarbyl group, H, OH or ═O, or represent a bond with an adjacent atom on the ring of the cyclic compound; or

(b) R¹ and R² are independently selected from —OH, ═O, and —OC(O)R′, wherein R′ is a hydrocarbyl group

R³ is selected from —OH, ═O, a substituent comprising an —OH group and —OC(O)R′, wherein R′ is a H or a hydrocarbyl group;

R⁴ and R⁵ are each independently selected from a hydrocarbyl group, H, OH, ═O, and —OC(O)R′, wherein R′ is a H or a hydrocarbyl group or wherein R⁴ and R⁵ represent a bond with an adjacent atom on the ring of the cyclic compound;

and wherein said compound comprises at least one ester group.

Such derivatives are described in more detail in pending applications WO 02/26060 and WO 02/26061, both filed on Sep. 27, 2001.

In a particularly preferred embodiment of the invention, the derivative of ascopyrone P is selected from ascopyrone M, ascopyrone T, ascopyrone T₁, ascopyrone T₂, and ascopyrone T₃, and mixtures thereof, the structures of which are shown below.

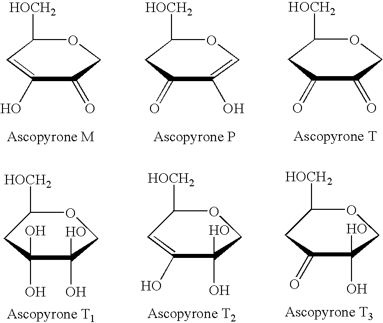

In a further preferred embodiment of the invention, the derivative of ascopyrone P is selected from the following:

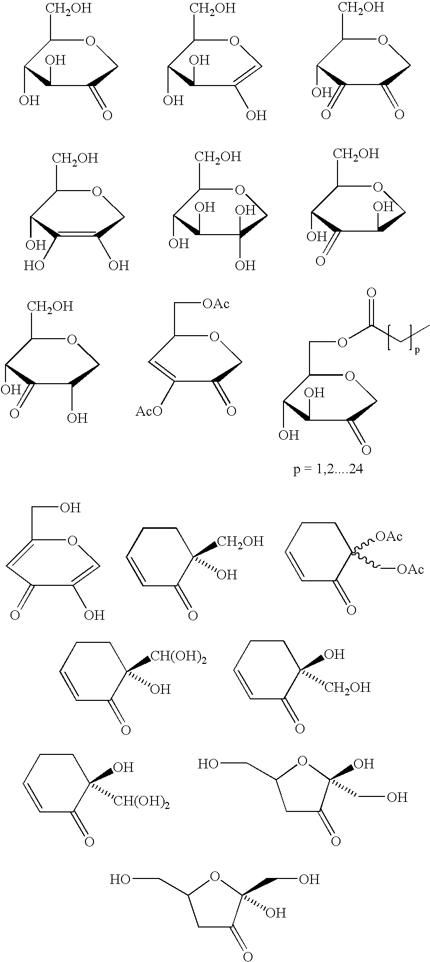

In one particularly preferred embodiment embodiment, the derivative of ascopyrone P is microthecin or echinosporin.

In another preferred embodiment, the process further comprises the use of isoamylase and/or pullalanase. Said isoamylase and/or pullalanase serve to increase the yield of 1,5-anhydro-D-fructose.

In yet another preferred embodiment, the process further comprises the use of one or more divalent metal salts, preferably NaCl or CaCl₂.

Preferably, the process has a reaction time of from about 1 to about 7 days, preferably from about 2 to about 5 days.

In one preferred embodiment, said α-1,4-glucan lyase and/or 1,5-anhydro-D-fructose dehydratase and/or pyranosone dehydratase and/or ascopyrone P synthase are in free form.

In another preferred embodiment, said α-1,4-glucan lyase and/or 1,5-anhydro-D-fructose dehydratase and/or pyranosone dehydratase and/or ascopyrone P synthase are immobilised on a support.

More preferably still, said α-1,4-glucan lyase and/or 1,5-anhydro-D-fructose dehydratase and/or pyranosone dehydratase and/or ascopyrone P synthase are immobilised on a succinimide-activated or a glutardiadehyde-activated solid support.

In yet another preferred embodiment, said α-1,4-glucan lyase and/or 1,5-anhydro-D-fructose dehydratase and/or pyranosone dehydratase and/or ascopyrone P synthase are held in membrane containers.

Preferably, said ascopyrone P or derivative thereof is purified by selective extraction.

Even more preferably, said ascopyrone P or derivative thereof is extracted with an organic solvent selected from acetonitrile, ethyl acetate, ethanol, propanol, isopropanol, acetone and butanol.

Even more preferably, said ascopyrone P or derivative thereof is concentrated under reduced pressure and optionally crystallised from an organic solvent.

In one preferred embodiment, said ascopyrone P or derivative thereof is purified by reverse phase or normal phase chromatography.

In another preferred embodiment, said ascopyrone P or derivative thereof is purified by ion exchange chromatography and/or gel filtration.

Preferably, said ascopyrone P or derivative thereof is further processed by freeze drying or spray drying.

In a preferred embodiment, step (II) comprises converting said 1,5-anhydro-D-fructose to ascopyrone P with ascopyrone P synthase and 1,5-anhydro-D-fructose dehydratase.

Preferably, said 1,5-anhydro-D-fructose dehydratase is characterised by one or more of the following:

(a) having a temperature optimum of from about 34 to 50° C.;

(b) having an optimal pH range of from about 5.9 to about 7.0;

(c) being stable in 50 mM sodium phosphate buffer (pH 7.0) containing 0.1 M NaCl for at least two weeks at 4° C.; or

(c) exhibiting enhanced activity in the presence of Mg²⁺, Ca²⁺ or Na²⁺ ions;

(d) being inhibited in the presence of one or more of ZnCl₂, EDTA or DTT.

In a particularly preferred embodiment, said 1,5-anhydro-D-fructose dehydratase is purified and characterised in accordance with pending PCT application [Number to be advised: Agent's Reference P11933WO, paragraphing priority from GB 0126165.0].

In another preferred embodiment, step (II) comprises converting said 1,5-anhydro-D-fructose to ascopyrone P with ascopyrone P synthase and pyranosone dehydratase.

Preferably, said pyranosone dehydratase is encoded by the nucleotide sequence set forth in SEQ. ID. No.1.

Preferably, said pyranosone dehydratase comprises at least one sequence selected from (i) to (xiii) below:

| | |
|---|---|
| (i) | KPHCEPEQPAALPLFQPQLVQGGRPDXYWVEAFPFRSDSSK or |
| | KPHXEPEQPAALPLFQPQLVV(Q)GGRPDXY; |
| (ii) | SDIQMFVNPYATTNNQSSXWTPVSLAKLDFPVAMHYADITK; |
| (iii) | VSWLENPGELR; |
| (iv) | DGVDCLWYDGAR; |
| (v) | PAGSPTGIVRAEWTRHVLDVFGXLXXK; |
| (vi) | HTGSIHQVVCADIDGDGEDEFLVAMMGADPPDFQRTGVWCYK; |
| (vii) | TEMEFLDVAGK; |
| (viii) | KLTLVVLPPFARLDVERNVSGVK; |
| (ix) | SMDELVAHNLFPAYVPDSVR; |
| (x) | NDATDGTPVLALLDLDGGPSPQAWNISHVPPGTDMYEIAHAK; |
| (xi) | TGSLVCARWPPVK; |
| (xii) | NQRVAGTHSPAAMGLTSRWAVTK; |
| (xiii) | GQITFRLPEAPDHGPLFLSVSAIRHQ; |

or a variant, homologue, fragment or derivative thereof, with the proviso that said pyranosone dehydratase does not contain both sequence (i) and sequence (xiv).

In one preferred embodiment, said pyranosone dehydratase comprises one sequence selected from sequences (i) to (xiii) above.

In another preferred embodiment, said pyranosone dehydratase comprises two sequences selected from sequences (i) to (xiii) above.

In another preferred embodiment, said pyranosone dehydratase comprises three sequences selected from sequences (i) to (xiii) above.

In another preferred embodiment, said pyranosone dehydratase comprises four sequences selected from sequences (i) to (xiii) above.

In another preferred embodiment, said pyranosone dehydratase comprises five sequences selected from sequences (i) to (xiii) above.

In another preferred embodiment, said pyranosone dehydratase comprises six sequences selected from sequences (i) to (xiii) above.

In another preferred embodiment, said pyranosone dehydratase comprises seven sequences selected from sequences (i) to (xiii) above.

In another preferred embodiment, said pyranosone dehydratase comprises eight sequences selected from sequences (i) to (xiii) above.

In another preferred embodiment, said pyranosone dehydratase comprises nine sequences selected from sequences (i) to (xiii) above.

In another preferred embodiment, said pyranosone dehydratase ten sequences selected from sequences (i) to (xiii) above.

In another preferred embodiment, said pyranosone dehydratase comprises eleven sequences selected from sequences (i) to (xiii) above.

In another preferred embodiment, said pyranosone dehydratase comprises twelve sequences selected from sequences (i) to (xiii) above.

In another preferred embodiment, said pyranosone dehydratase comprises thirteen sequences selected from sequences (i) to (xiii) above.

In a particularly preferred embodiment, the pyranosone dehydratase is purified, characterised and has an amino acid sequence in accordance with pending PCT application [Number to be advised: Agent's Reference P11937WO, paragraphing priority from GB 0126164.3].

In a further preferred embodiment, said ascopyrone P synthase is characterised by one or more of the following:

(a) having an optimim temperature range of 25 to 50° C.;

(b) having an optimal pH range of from about 4.5 to 7.5;

(c) being stable in 50 mM sodium phosphate buffer (pH 7.0) containing 0.1 M NaCl for at least one month at 4° C.; or

(d) comprising at least one amino acid sequence selected from (i) AINLPFSNWAX(or C)TI and (ii) EYGRTFFTRYDYENVD.

In a particularly preferred embodiment, the ascopyrone P synthase is purified, characterised and has an amino acid sequence in accordance with pending PCT application [Number to be advised: Agent's Reference P12627WO, paragraphing priority from GB 0126163.5].

Preferably, said α-1,4-glucan lyase, ascopyrone P synthase, 1,5-anhydro-D-fructose dehydratase and pyranosone dehydratase have a purity of greater than 90%.

More preferably, said α-1,4-glucan lyase, ascopyrone P synthase, 1,5-anhydro-D-fructose dehydratase and pyranosone dehydratase are in pure or substantially pure form.

Advantages

The present invention provides an improved method of preparing ascopyrone P, and derivatives thereof. In particular the invention alleviates some of the problems associated with prior art preparations, and provides a process suitable for the large scale preparation of ascopyrone P and its derivatives. The described process is advantageous in view of the potential commercial applications of ascopyrone P and related compounds as antioxidants, antibrowning agents and antimicrobial agents. As used herein, the term “enzyme” or “enzymes” refers to one or more of α-1,4-glucan lyase, ascopyrone P synthase, 1,5-anhydro-D-fructose dehydratase and pyranosone dehydratase.

Isolated

In one aspect, preferably the enzymes are in an isolated form. The term “isolated” means that the enzyme is not in its natural environment (i.e. as found in nature). Typically the term “isolated” means that the enzyme is at least substantially free from at least one other component with which the enzyme is naturally associated in nature and as found in nature. Here, the enzyme may be separated from at least one other component with which it is naturally associated.

Purified

In one aspect, preferably the enzymes are in a purified form. The term “purified” also means that the enzyme is not in its natural environment (i.e. as found in nature). Typically the term “purified” means that the enzyme is at least substantially separated from at least one other component with which the enzyme is naturally associated in nature and as found in nature.

Nucleotide Sequence

The present invention encompasses nucleotide sequences encoding enzymes having the specific properties as defined herein. The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or antisense strand.

The term “nucleotide sequence” in relation to the present invention includes genomic DNA, cDNA, synthetic DNA, and RNA. Preferably it means DNA, more preferably cDNA for the coding sequence of the present invention.

In a preferred embodiment, the nucleotide sequence per se of the present invention does not cover the native nucleotide sequence according to the present invention in its natural environment when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence of the present invention can be isolated and/or purified post expression of a nucleotide sequence in its native organism. Preferably, however, the amino acid sequence of the present invention may be expressed by a nucleotide sequence in its native organism but wherein the nucleotide sequence is not under the control of the promoter with which it is naturally associated within that organism.

Typically, the nucleotide sequence of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al (1980) Nuc Acids Res Symp Ser 215-23 and Horn T et al (1980) Nuc Acids Res Symp Ser 225-232).

Preparation of the Nucleotide Sequence

A nucleotide sequence encoding either an enzyme which has the specific properties as defined herein or an enzyme which is suitable for modification may be identified and/or isolated and/or purified from any cell or organism producing said enzyme. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al (1981) Tetrahedron Letters 22, p 1859-1869, or the method described by Matthes et al (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al (Science (1988) 239, pp 487-491).

Amino Acid Sequences

As mentioned above, information relating to AFDH, APS and pyranosone dehydratase is described in co-pending PCT applications [Numbers to be advised: Agent's Reference P11933WO (paragraphing priority from GB 0126165.0), P12627WO (paragraphing priority from GB 0126163.5) and P11937WO (paragraphing priority from GB 0126164.3) respectively], the contents of which are hereby incorporated by reference.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The enzymes of the present invention may be used in conjunction with other enzymes. Thus the present invention also covers a combination of enzymes wherein the combination comprises the enzyme of the present invention and another enzyme, which may be another enzyme according to the present invention. This aspect is discussed in a later section.

Preferably the enzyme is not a native enzyme. In this regard, the term “native enzyme” means an entire enzyme that is in its native environment and when it has been expressed by its native nucleotide sequence.

Variants/Homologues/Derivatives

The present invention also encompasses the use of variants, homologues and derivatives of any amino acid sequence of an enzyme of the present invention or of any nucleotide sequence encoding such an enzyme. Here, the term “homologue” means an entity having a certain homology with the subject amino acid sequences and the subject nucleotide sequences. Here, the term “homology” can be equated with “identity”.

In the present context, an homologous sequence is taken to include an amino acid sequence which may be at least 75, 80, 85 or 90% identical, preferably at least 95, 96, 97, 98 or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 40, 50, 60, 70, 75, 80, 85 or 90% identical, preferably at least 95, 96, 97, 98 or 99% identical to a nucleotide sequence encoding an enzyme of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (Devereux et al 1984 Nuc. Acids Research 12 p387). Examples of other software than can perform sequence comparisons include, but are not limited to, the BLAST package (see Ausubel et al 1999 Short Protocols in Molecular Biology, 4^{th} Ed—Chapter 18), FASTA (Altschul et al 1990 J. Mol. Biol. 403-410) and the GENEWORKS suite of comparison tools. Both BLAST and FASTA are available for offline and online searching (see Ausubel et al 1999, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8 and tatiana@ncbi.nlm.nih.gov).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Alternatively, percentage homologies may be calculated using the multiple alignment feature in DNASIS™ (Hitachi Software), based on an algorithm, analogous to CLUSTAL (Higgins DG & Sharp PM (1988), Gene 73(1), 237-244).

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-α-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Suitable fragments will be at least 5, e.g. 10, 12, 15 or 20 amino acids in length. They may also be less than 100, 75 or 50 amino acids in length. They may contain one or more (e.g. 5, 10, 15 or 20) substitutions, deletions or insertions, including conserved substitutions.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any homologue, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The present invention also encompasses polynucleotides which have undergone molecular evolution via random processes, selection mutagenesis or in vitro recombination. As a non-limiting example, it is possible to produce numerous site directed or random mutations into a nucleotide sequence, either in vivo or in vitro, and to subsequently screen for improved functionality of the encoded polypeptide by various means. In addition, mutations or natural variants of a polynucleotide sequence can be recombined with either the wildtype or other mutations or natural variants to produce new variants. Such new variants can also be screened for improved functionality of the encoded polypeptide. The production of new preferred variants can be achieved by various methods well established in the art, for example the Error Threshold Mutagenesis (WO 92/18645), oligonucleotide mediated random mutagenesis (U.S. Pat. No. 5,723,323), DNA shuffling (U.S. Pat. No. 5,605,793), exo-mediated gene assembly WO 00/58517. The application of these and similar random directed molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. This will involve making a pair of primers (e.g. of about 15 to 30 nucleotides) flanking a region of the lipid targeting sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Biologically Active

Preferably, the variant sequences etc. are at least as biologically active as the sequences presented herein.

As used herein “biologically active” refers to a sequence having a similar structural function (but not necessarily to the same degree), and/or similar regulatory function (but not necessarily to the same degree), and/or similar biochemical function (but not necessarily to the same degree) of the naturally occurring sequence.

Isozymes

The enzymes of the present invention may exist in the form of one or more different isozymes. As used herein, the term “isozyme” encompasses variants of the polypeptide that catalyse the same reaction, but differ from each other in properties such as substrate affinity and maximum rates of enzyme-substrate reaction. Owing to differences in amino acid sequence, isozymes can be distinguished by techniques such as electrophoresis or isoelectric focusing. Different tissues often have different isoenzymes. The sequence differences generally confer different enzyme kinetic parameters that can sometimes be interpreted as fine tuning to the specific requirements of the cell types in which a particular isoenzyme is found.

Isoforms

The present invention also encompasses different isoforms of the enzymes described herein. The term “isoform” refers to a protein having the same function (namely pyranosone dehydratase activity), which has a similar or identical amino acid sequence, but which is the product of a different gene.

Hybridisation

The present invention also encompasses sequences that are complementary to the sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃ citrate pH 7.0}) to the nucleotide sequences presented herein.

More preferably, the term “variant” encompasses sequences that are complementary to sequences that are capable of hybridising under high stringent conditions (e.g. 65° C. and 0.1×SSC {1×SSC=0.15 M NaCl, 0.015 M Na₃ citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under stringent conditions (e.g. 50° C. and 0.2×SSC).

In a more preferred aspect, the present invention covers nucleotide sequences that can hybridise to the nucleotide sequence of the present invention, or the complement thereof, under high stringent conditions (e.g. 65° C. and 0.1×SSC).

Site-Directed Mutagenesis

Once an enzyme-encoding nucleotide sequence has been isolated, or a putative enzyme-encoding nucleotide sequence has been identified, it may be desirable to mutate the sequence in order to prepare an enzyme of the present invention.

Mutations may be introduced using synthetic oligonucleotides. These oligonucleotides contain nucleotide sequences flanking the desired mutation sites.

A suitable method is disclosed in Morinaga et al (Biotechnology (1984) 2, p646-649), wherein a single-stranded gap of DNA, the enzyme-encoding sequence, is created in a vector carrying the enzyme gene. The synthetic nucleotide, bearing the desired mutation, is then annealed to a homologous portion of the single-stranded DNA. The remaining gap is then filled in with DNA polymerase I (Klenow fragment) and the construct is ligated using T4 ligase.

U.S. Pat. No. 4,760,025 discloses the introduction of oligonucleotides encoding multiple mutations by performing minor alterations of the cassette. However, an even greater variety of mutations can be introduced at any one time by the above mentioned Morinaga method, because a multitude of oligonucleotides, of various lengths, can be introduced.

Another method of introducing mutations into enzyme-encoding nucleotide sequences is described in Nelson and Long (Analytical Biochemistry (1989), 180, p 147-151). This method involves the 3-step generation of a PCR fragment containing the desired mutation introduced by using a chemically synthesised DNA strand as one of the primers in the PCR reactions. From the PCR-generated fragment, a DNA fragment carrying the mutation may be isolated by cleavage with restriction endonucleases and reinserted into an expression plasmid.

By way of example, Sierks et al (Protein Eng (1989) 2, 621-625 and Protein Eng (1990) 3, 193-198) describes site-directed mutagenesis in Aspergillus glucoamylase.

Recombinant

In one aspect of the present invention the sequence is a recombinant sequence—i.e. a sequence that has been prepared using recombinant DNA techniques.

Synthetic

In one aspect of the present invention the sequence is a synthetic sequence—i.e. a sequence that has been prepared by in vitro chemical or enzymatic synthesis. It includes but is not limited to sequences made with optimal codon usage for host organisms, such as the the methylotrophic yeasts Pichia and Hansenula.

Expression of Enzymes

The nucleotide sequence for use in the present invention can be incorporated into a recombinant replicable vector. The vector may be used to replicate and express the nucleotide sequence, in enzyme form, in and/or from a compatible host cell. Both homologous and heterologous expression is contemplated.

For homologous expression, preferably the gene of interest or nucleotide sequence of interest is not in its naturally occurring genetic context. In the case where the gene of interest or nucleotide sequence of interest is in its naturally occurring genetic context, preferably expression is driven by means other than or in addition to its naturally occurring expression mechanism; for example, by overexpressing the gene of interest by genetic intervention.

Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above.

The enzyme produced by a host recombinant cell by expression of the nucleotide sequence may be secreted or may be contained intracellularly depending on the sequence and/or the vector used. The coding sequences can be designed with signal sequences which direct secretion of the substance coding sequences through a particular prokaryotic or eukaryotic cell membrane.

Expression Vector

The term “expression vector” means a construct capable of in vivo or in vitro expression.

Preferably, the expression vector is incorporated in the genome of a suitable host organism. The term “incorporated” preferably covers stable incorporation into the genome.

The host organism can be the same or different to the gene of interest source organism, giving rise to homologous and heterologous expression respectively.

Preferably, the vector of the present invention comprises a construct according to the present invention. Alternatively expressed, preferably the nucleotide sequence of the present invention is present in a vector and wherein the nucleotide sequence is operably linked to regulatory sequences such that the regulatory sequences are capable of providing the expression of the nucleotide sequence by a suitable host organism, i.e. the vector is an expression vector.

The vectors of the present invention may be transformed into a suitable host cell as described below to provide for expression of a polypeptide of the present invention. Thus, in a further aspect the invention provides a process for preparing polypeptides for subsequent use according to the present invention which comprises cultivating a host cell transformed or transfected with an expression vector under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter. The choice of vector will often depend on the host cell into which it is to be introduced.

The vectors of the present invention may contain one or more selectable marker genes. The most suitable selection systems for industrial micro-organisms are those formed by the group of selection markers which do not require a mutation in the host organism. Suitable selection markers may be the dal genes from B. subtilis or B. licheniformis, or one which confers antibiotic resistance such as ampicillin, kanamycin, chloramphenicol or tetracyclin resistance. Alternative selection markers may be the Aspergillus selection markers such as amdS, argB, niaD and sC, or a marker giving rise to hygromycin resistance. Examples of other fungal selection markers are the genes for ATP synthetase, subunit 9 (oliC), orotidine-5′-phosphate-decarboxylase (pvrA), phleomycin and benomyl resistance (benA). Examples of non-fungal selection markers are the bacterial G418 resistance gene (this may also be used in yeast, but not in filamentous fungi), the ampicillin resistance gene (E. coli), the neomycin resistance gene (Bacillus) and the E. coli uidA gene, coding for β-glucuronidase (GUS). Further suitable selection markers include the dal genes from B. subtilis or B. licheniformis. Alternatively, the selection may be accomplished by co-transformation (as described in WO91/17243).

Vectors may be used in vitro, for example for the production of RNA or used to transfect or transform a host cell.

Thus, nucleotide sequences for use according to the present invention can be incorporated into a recombinant vector (typically a replicable vector), for example a cloning or expression vector. The vector may be used to replicate the nucleic acid in a compatible host cell. Thus in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector. The vector may be recovered from the host cell. Suitable host cells are described below in connection with expression vectors.

The procedures used to ligate a DNA construct of the invention encoding an enzyme which has the specific properties as defined herein, and the regulatory sequences, and to insert them into suitable vectors containing the information necessary for replication, are well known to persons skilled in the art (for instance see Sambrook et al Molecular Cloning: A laboratory Manual, 2^{nd} Ed. (1989)).

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

The expression vector typically includes the components of a cloning vector, such as, for example, an element that permits autonomous replication of the vector in the selected host organism and one or more phenotypically detectable markers for selection purposes. The expression vector normally comprises control nucleotide sequences encoding a promoter, operator, ribosome binding site, translation initiation signal and optionally, a repressor gene or one or more activator genes. Additionally, the expression vector may comprise a sequence coding for an amino acid sequence capable of targeting the amino acid sequence to a host cell organelle such as a peroxisome or to a particular host cell compartment. In the present context, the term “expression signal” includes any of the above control sequences, repressor or activator sequences. For expression under the direction of control sequences, the nucleotide sequence is operably linked to the control sequences in proper manner with respect to expression.

Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of the enzyme of the present invention. In eukaryotes, polyadenylation sequences may be operably connected to the nucleotide sequence encoding the enzyme.

Preferably, the nucleotide sequence of the present invention may be operably linked to at least a promoter.

Aside from the promoter native to the gene encoding the nucleotide sequence of the present invention, other promoters may be used to direct expression of the polypeptide of the present invention. The promoter may be selected for its efficiency in directing the expression of the nucleotide sequence of the present invention in the desired expression host.

In another embodiment, a constitutive promoter may be selected to direct the expression of the desired nucleotide sequence of the present invention. Such an expression construct may provide additional advantages since it circumvents the need to culture the expression hosts on a medium containing an inducing substrate.

Examples of suitable promoters for directing the transcription of the nucleotide sequence in a bacterial host include the promoter of the lac operon of E. coli, the Streptomyces coelicolor agarase gene dagA promoters, the promoters of the Bacillus licheniformis α-amylase gene (amyL), the promoters of the Bacillus stearothermophilus maltogenic amylase gene (amyM), the promoters of the Bacillus amyloliquefaciens α-amylase gene (amyQ), the promoters of the Bacillus subtilis xylA and xylB genes and a promoter derived from a Lactococcus sp.-derived promoter including the P170 promoter. When the nucleotide sequence is expressed in a bacterial species such as E. coli, a suitable promoter can be selected, for example, from a bacteriophage promoter including a T7 promoter and a phage lambda promoter.

For transcription in a fungal species, examples of useful promoters are those derived from the genes encoding the, Aspergillus oryzae TAKA amylase, Rhizomucor miehei aspartic proteinase, Aspergillus niger neutral α-amylase, A. niger acid stable α-amylase, A. niger glucoamylase, Rhizomucor miehei lipase, Aspergillus oryzae alkaline protease, Aspergillus oryzae triose phosphate isomerase or Aspergillus nidulans acetamidase.

Examples of strong constitutive and/or inducible promoters which are preferred for use in fungal expression hosts are those which are obtainable from the fungal genes for xylanase (xlnA), phytase, ATP-synthetase, subunit 9 (oliC), triose phosphate isomerase (tpi), alcohol dehydrogenase (AdhA), α-amylase (amy), amyloglucosidase (AG—from the glaA gene), acetamidase (amdS) and glyceraldehyde-3-phosphate dehydrogenase (gpd) promoters. Other examples of useful promoters for transcription in a fungal host are those derived from the gene encoding A. oryzae TAKA amylase, the TPI (triose phosphate isomerase) promoter from S. cerevisiae (Alber et al (1982) J. Mol. Appl. Genet. 1, p419-434), Rhizomucor miehei aspartic proteinase, A. niger neutral α-amylase, A. niger acid stable α-amylase, A. niger glucoamylase, Rhizomucor miehei lipase, A. oryzae alkaline protease, A. oryzae triose phosphate isomerase or A. nidulans acetamidase.

Examples of suitable promoters for the expression in a yeast species include but are not limited to the Gal 1 and Gal 10 promoters of Saccharomyces cerevisiae and the Pichia pastoris AOX1 or AOX2 promoters.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the nucleotide sequence of the present invention. For example, suitable other sequences include the Sh1-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat 1987 Gene 217, 217-225 and Dawson 1993 Plant Mol. Biol. 23: 97).

Constructs

The term “construct”—which is synonymous with terms such as “conjugate”, “cassette” and “hybrid”— includes a nucleotide sequence for use according to the present invention directly or indirectly attached to a promoter. An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker which allows for the selection of the genetic construct in, for example, a bacterium, preferably of the genus Bacillus, such as Bacillus subtilis, or plants into which it has been transferred. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

For some applications, preferably the construct of the present invention comprises at least the nucleotide sequence of the present invention operably linked to a promoter.

Host Cells

The term “host cell”—in relation to the present invention includes any cell that comprises either the nucleotide sequence or an expression vector as described above and which is used in the recombinant production of an enzyme having the specific properties as defined herein. The nucleotide of interest may be homologous or heterologous to the host cell.

Thus, a further embodiment of the present invention provides host cells transformed or transfected with a nucleotide sequence that expresses the enzyme of the present invention. Preferably said nucleotide sequence is carried in a vector for the replication and expression of the nucleotide sequence. The cells will be chosen to be compatible with the said vector and may for example be prokaryotic (for example bacterial), fungal, yeast or plant cells.

Examples of suitable bacterial host organisms are gram positive bacterial species such as Bacillaceae including Bacillus subtilis, Bacillus licheniformis, Bacillus lentus, Bacillus brevis, Bacillus stearothermophilus, Bacillus alkalophilus, Bacillus amyloliquefaciens, Bacillus coagulans, Bacillus lautus, Bacillus megaterium and Bacillus thuringiensis, Streptomyces species such as Streptomyces murinus, lactic acid bacterial species including Lactococcus spp. such as Lactococcus lactis, Lactobacillus spp. including Lactobacillus reuteri, Leuconostoc spp., Pediococcus spp. and Streptococcus spp. Alternatively, strains of a gram-negative bacterial species belonging to Enterobacteriaceae including E. coli, or to Pseudomonadaceae can be selected as the host organism.

The gram negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, Gram positive bacteria from the genus Bacillus, such as B. subtilis, B. licheniformis, B. lentus, B. brevis, B. stearothermophilus, B. alkalophilus, B. amyloliquefaciens, B. coagulans, B. circulans, B. lautus, B. megaterium, B. thuringiensis, Streptomyces lividans or S. murinus, may be very suitable as heterologous hosts because of their capability to secrete proteins into the culture medium. Other bacteria that may be suitable as hosts are those from the genera Streptomyces and Pseudomonas.

Depending on the nature of the nucleotide sequence encoding the enzyme of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Typical fungal expression hosts may be selected from Aspergillus niger, Aspergillus niger var. tubigenis, Aspergillus niger var. awamori, Aspergillus aculeatis, Aspergillus nidulans, Aspergillus oryzae, Trichoderma reesei, Bacillus subtilis, Bacillus licheniformis, Bacillus amyloliquefaciens, Kluyveromyces lactis and Saccharomyces cerevisiae.

Suitable filamentous fungus may be for example a strain belonging to a species of Aspergillus, such as Aspergillus oryzae or Aspergillus niger, or a strain of Fusarium oxysporium, Fusarium graminearum (in the perfect state named Gribberella zeae, previously Sphaeria zeae, synonym with Gibberella roseum and Gibberella roseum f. sp. Cerealis), or Fusarium sulphureum (in the perfect state named Gibberella puricaris, synonym with Fusarium trichothercioides, Fusarium bactridioides, Fusarium sambucium, Fusarium roseum and Fusarium roseum var. graminearum), Fusarium cerealis (synonym with Fusarium crokkwellnse) or Fusarium venenatum.

Suitable yeast organisms may be selected from the species of Kluyveromyces, Saccharomyces or Schizosaccharomyces, e.g. Saccharomyces cerevisiae, or Hansenula (disclosed in UK Patent Application No. 9927801.2).

The use of suitable host cells—such as yeast, fungal and plant host cells—may provide for post-translational modifications (e.g. myristoylation, glycosylation, truncation, lapidation and tyrosine, serine or threonine phosphorylation) as may be needed to confer optimal biological activity on recombinant expression products of the present invention.

The host cell may be a protease deficient or protease minus strain. This may for example be the protease deficient strain Aspergillus oryzae JaL 125 having the alkaline protease gene named “alp” deleted. This strain is described in WO97/35956.

Organism

The term “organism” in relation to the present invention includes any organism that could comprise the nucleotide sequence coding for the enzyme according to the present invention and/or products obtained therefrom, and/or wherein a promoter can allow expression of the nucleotide sequence according to the present invention when present in the organism.

Suitable organisms may include a prokaryote, fungus, yeast or a plant.

The term “transgenic organism” in relation to the present invention includes any organism that comprises the nucleotide sequence coding for the enzyme according to the present invention and/or the products obtained therefrom, and/or wherein a promoter can allow expression of the nucleotide sequence according to the present invention within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the enzyme according to the present invention, constructs according to the present invention, vectors according to the present invention, plasmids according to the present invention, cells according to the present invention, tissues according to the present invention, or the products thereof. For example the transgenic organism can also comprise the nucleotide sequence coding for the enzyme of the present invention under the control of a heterologous promoter.

Transformation of Host Cells/Organism

As indicated earlier, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc. If a prokaryotic host is used then the nucleotide sequence may need to be suitably modified before transformation—such as by removal of introns.

Filamentous fungi cells may be transformed by a process involving protoplast formation and transformation of the protoplasts followed by regeneration of the cell wall in a manner known. The use of Aspergillus as a host microorganism is described in EP 0 238 023.

Another host organism can be a plant. The basic principle in the construction of genetically modified plants is to insert genetic information in the plant genome so as to obtain a stable maintenance of the inserted genetic material. Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27). Further teachings on plant transformation may be found in EP-A-0449375.

General teachings on the transformation of fungi, yeasts and plants are presented in following sections.

Transformed Fungus

A host organism may be a fungus—such as a mold. Examples of suitable such hosts include any member belonging to the genera Phanerochaete, Thermomyces, Acremonium, Aspergillus, Penicillium, Mucor, Neurospora, Trichoderma and the like—such as Thermomyces lanuginosis, Acremonium chrysogenum, Aspergillus niger, Aspergillus oryzae, Aspergillus awamori, Penicillinum chrysogenem, Mucor javanious, Neurospora crassa, Trichoderma viridae, Phanerochaete chrysosporium, and the like.

In one embodiment, the host organism may be a filamentous fungus.

For almost a century, filamentous fungi have been widely used in many types of industry for the production of organic compounds and enzymes. For example, traditional Japanese koji and soy fermentations have used Aspergillus sp. Also, in this century Aspergillus niger has been used for production of organic acids particular citric acid and for production of various enzymes for use in industry.

There are two major reasons why filamentous fungi have been so widely used in industry. First filamentous fungi can produce high amounts of extracellular products, for example enzymes and organic compounds such as antibiotics or organic acids. Second filamentous fungi can grow on low cost substrates such as grains, bran, beet pulp etc. The same reasons have made filamentous fungi attractive organisms as hosts for heterologous expression according to the present invention.

In order to prepare the transgenic Aspergillus, expression constructs are prepared by inserting the nucleotide sequence according to the present invention into a construct designed for expression in filamentous fungi.

Several types of constructs used for heterologous expression have been developed. These constructs preferably contain one or more of: a signal sequence which directs the amino acid sequence to be secreted, typically being of fungal origin, and a terminator (typically being active in fungi) which ends the expression system.

Another type of expression system has been developed in fungi where the nucleotide sequence according to the present invention can be fused to a smaller or a larger part of a fungal gene encoding a stable protein. This can stabilise the amino acid sequence. In such a system a cleavage site, recognised by a specific protease, can be introduced between the fungal protein and the amino acid sequence, so the produced fusion protein can be cleaved at this position by the specific protease thus liberating the amino acid sequence. By way of example, one can introduce a site which is recognised by a KEX-2 like peptidase found in at least some Aspergilli. Such a fusion leads to cleavage in vivo resulting in production of the expressed product and not a larger fusion protein.

Heterologous expression in Aspergillus has been reported for several genes coding for bacterial, fungal, vertebrate and plant proteins. The proteins can be deposited intracellularly if the nucleotide sequence according to the present invention is not fused to a signal sequence. Such proteins will accumulate in the cytoplasm and will usually not be glycosylated which can be an advantage for some bacterial proteins. If the nucleotide sequence according to the present invention is equipped with a signal sequence the protein will accumulate extracellularly.

With regard to product stability and host strain modifications, some heterologous proteins are not very stable when they are secreted into the culture fluid of fungi. Most fungi produce several extracellular proteases which degrade heterologous proteins. To avoid this problem special fungal strains with reduced protease production have been used as host for heterologous production.

Teachings on transforming filamentous fungi are reviewed in U.S. Pat. No. 5,741,665 which states that standard techniques for transformation of filamentous fungi and culturing the fungi are well known in the art. An extensive review of techniques as applied to N. crassa is found, for example in Davis and de Serres, Methods Enzymol (1971) 17A:79-143. Standard procedures are generally used for the maintenance of strains and the preparation of conidia. Mycelia are typically grown in liquid cultures for about 14 hours (25° C.), as described in Lambowitz et al., J Cell Biol (1979) 82:17-31. Host strains can generally be grown in either Vogel's or Fries minimal medium supplemented with the appropriate nutrient(s), such as, for example, any one or more of: his, arg, phe, tyr, trp, p-aminobenzoic acid, and inositol.

Further teachings on transforming filamentous fungi are reviewed in U.S. Pat. No. 5,674,707 which states that once a construct has been obtained, it can be introduced either in linear form or in plasmid form, e.g., in a pUC-based or other vector, into a selected filamentous fungal host using a technique such as DNA-mediated transformation, electroporation, particle gun bombardment, protoplast fusion and the like. In addition, Ballance 1991 (ibid) states that transformation protocols for preparing transformed fungi are based on preparation of protoplasts and introduction of DNA into the protoplasts using PEG and Ca²⁺ ions. The transformed protoplasts then regenerate and the transformed fungi are selected using various selective markers.

To allow for selection of the resulting transformants, the transformation typically also involves a selectable gene marker which is introduced with the expression cassette, either on the same vector or by co-transformation, into a host strain in which the gene marker is selectable. Various marker/host systems are available, including the pyrG, argB and niaD genes for use with auxotrophic strains of Aspergillus nidulans; pyrG and argB genes for Aspergillus oryzae auxotrophs; pyrG, trpc and niaD genes for Penicillium chrysogenum auxotrophs; and the argB gene for Trichoderma reesei auxotrophs. Dominant selectable markers including amdS, oliC, hyg and phleo are also now available for use with such filamentous fungi as A. niger, A. oryzae, A. ficuum, P. chrysogenum, Cephalosporium acremonium, Cochliobolus heterostrophus, Glomerella cingulata, Fulvia fulva and Leptosphaeria maculans (for a review see Ward in Modem Microbial Genetics, 1991, Wiley-Liss, Inc., at pages 455-495). A commonly used transformation marker is the amdS gene of A. nidulans which in high copy number allows the fungus to grow with acrylamide as the sole nitrogen source.

For the transformation of filamentous fungi, several transformation protocols have been developed for many filamentous. Among the markers used for transformation are a number of auxotrophic markers such as argB, trpC, niaD and pyrG, antibiotic resistance markers such as benomyl resistance, hygromycin resistance and phleomycin resistance.

In one aspect, the host organism can be of the genus Aspergillus, such as Aspergillus niger.

A transgenic Aspergillus according to the present invention can also be prepared by following the teachings of Rambosek, J. and Leach, J. 1987 (Recombinant DNA in filamentous fungi: Progress and Prospects. CRC Crit. Rev. Biotechnol. 6:357-393), Davis R. W. 1994 (Heterologous gene expression and protein secretion in Aspergillus. In: Martinelli S. D., Kinghom J. R.(Editors) Aspergillus: 50 years on. Progress in industrial microbiology vol 29. Elsevier Amsterdam 1994. pp 525-560), Ballance, D. J. 1991 (Transformation systems for Filamentous Fungi and an Overview of Fungal Gene structure. In: Leong, S. A., Berka R. M. (Editors) Molecular Industrial Mycology. Systems and Applications for Filamentous Fungi. Marcel Dekker Inc. New York 1991. pp 1-29) and Turner G. 1994 (Vectors for genetic manipulation. In: Martinelli S. D., Kinghorn J. R. (Editors) Aspergillus: 50 years on. Progress in industrial microbiology vol 29. Elsevier Amsterdam 1994. pp. 641-666).

Transformed Yeast

In another embodiment the transgenic organism can be a yeast.

In this regard, yeast have also been widely used as a vehicle for heterologous gene expression.

By way of example, the species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401-429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence of the present invention into a construct designed for expression in yeast. Several types of constructs used for heterologous expression have been developed. The constructs may contain a promoter active in yeast, such as a promoter of yeast origin, such as the GAL1 promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces according to the present invention can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells may be selected using various selective markers. Among the markers used for transformation are a number of auxotrophic markers such as LEU2, HIS4 and TRP1, and dominant antibiotic resistance markers such as aminoglycoside antibiotic markers, eg G418.

Transformed Plants/Plant Cells

A preferred host organism suitable for the present invention is a plant.

In this respect, the basic principle in the construction of genetically modified plants is to insert genetic information in the plant genome so as to obtain a stable maintenance of the inserted genetic material.

Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Even though the promoter of the present invention is not disclosed in EP-B-0470145 and CA-A-2006454, those two documents do provide some useful background commentary on the types of techniques that may be employed to prepare transgenic plants according to the present invention. Some of these background teachings are now included in the following commentary.

The basic principle in the construction of genetically modified plants is to insert genetic information in the plant genome so as to obtain a stable maintenance of the inserted genetic material.

Thus, in one aspect, the present invention relates to a vector system which carries a nucleotide sequence or construct according to the present invention and which is capable of introducing the nucleotide sequence or construct into the genome of an organism, such as a plant.

The vector system may comprise one vector, but it can comprise two vectors. In the case of two vectors, the vector system is normally referred to as a binary vector system. Binary vector systems are described in further detail in Gynheung An et al. (1980), Binary Vectors, Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes An et al. (1986), Plant Physiol 81, 301-305 and Butcher D. N. et al. (1980), Tissue Culture Methods for Plant Pathologists, eds.: D. S. Ingrams and J. P. Helgeson, 203-208.

Several different Ti and Ri plasmids have been constructed which are suitable for the construction of the plant or plant cell constructs described above. A non-limiting example of such a Ti plasmid is pGV3850.

The nucleotide sequence or construct of the present invention should preferably be inserted into the Ti-plasmid between the terminal sequences of the T-DNA or adjacent a T-DNA sequence so as to avoid disruption of the sequences immediately surrounding the T-DNA borders, as at least one of these regions appear to be essential for insertion of modified T-DNA into the plant genome.

As will be understood from the above explanation, if the organism is a plant, then the vector system of the present invention is preferably one which contains the sequences necessary to infect the plant (e.g. the vir region) and at least one border part of a T-DNA sequence, the border part being located on the same vector as the genetic construct. Preferably, the vector system is an Agrobacterium tumefaciens Ti-plasmid or an Agrobacterium rhizogenes Ri-plasmid or a derivative thereof, as these plasmids are well-known and widely employed in the construction of transgenic plants, many vector systems exist which are based on these plasmids or derivatives thereof.

In the construction of a transgenic plant the nucleotide sequence or construct of the present invention may be first constructed in a micro-organism in which the vector can replicate and which is easy to manipulate before insertion into the plant. An example of a useful micro-organism is E coli., but other micro-organisms having the above properties may be used. When a vector of a vector system as defined above has been constructed in E. coli. it is transferred, if necessary, into a suitable Agrobacterium strain, e.g. Agrobacterium tumefaciens. The Ti-plasmid harbouring the nucleotide sequence or construct of the invention is thus preferably transferred into a suitable Agrobacterium strain, e.g. A. tumefaciens, so as to obtain an Agrobacterium cell harbouring the nucleotide sequence or construct of the invention, which DNA is subsequently transferred into the plant cell to be modified.

As reported in CA-A-2006454, a large amount of cloning vectors are available which contain a replication system in E. coli and a marker which allows a selection of the transformed cells. The vectors contain for example pBR 322, the pUC series, the M13 mp series, pACYC 184 etc.

In this way, the nucleotide or construct of the present invention can be introduced into a suitable restriction position in the vector. The contained plasmid is used for the transformation in E. coli. The E. coli cells are cultivated in a suitable nutrient medium and then harvested and lysed. The plasmid is then recovered. As a method of analysis there is generally used sequence analysis, restriction analysis, electrophoresis and further biochemical-molecular biological methods. After each manipulation, the used DNA sequence can be restricted and connected with the next DNA sequence. Each sequence can be cloned in the same or different plasmid.

After each introduction method of the desired promoter or construct or nucleotide sequence according to the present invention in the plants the presence and/or insertion of further DNA sequences may be necessary. If, for example, for the transformation the Ti- or Ri-plasmid of the plant cells is used, at least the right boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:1-46; and An et al., EMBO J. (1985) 4:277-284.

Direct infection of plant tissues by Agrobacterium is a simple technique which has been widely employed and which is described in Butcher D. N. et al. (1980), Tissue Culture Methods for Plant Pathologists, eds.: D. S. Ingrams and J. P. Helgeson, 203-208. For further teachings on this topic see Potrykus (Annu Rev Plant Physiol Plant Mol Biol [1991] 42:205-225) and Christou (Agro-Food-Industry Hi-Tech March/April 1994 17-27). With this technique, infection of a plant may be done on a certain part or tissue of the plant, i.e. on a part of a leaf, a root, a stem or another part of the plant.

Typically, with direct infection of plant tissues by Agrobacterium carrying the promoter and/or the GOI, a plant to be infected is wounded, e.g. by cutting the plant with a razor or puncturing the plant with a needle or rubbing the plant with an abrasive. The wound is then inoculated with the Agrobacterium. The inoculated plant or plant part is then grown on a suitable culture medium and allowed to develop into mature plants.

When plant cells are constructed, these cells may be grown and maintained in accordance with well-known tissue culturing methods such as by culturing the cells in a suitable culture medium supplied with the necessary growth factors such as amino acids, plant hormones, vitamins, etc. Regeneration of the transformed cells into genetically modified plants may be accomplished using known methods for the regeneration of plants from cell or tissue cultures, for example by selecting transformed shoots using an antibiotic and by subculturing the shoots on a medium containing the appropriate nutrients, plant hormones, etc.

Other techniques for transforming plants include ballistic transformation, the silicon whisker carbide technique (see Frame B R, Drayton P R, Bagnaall S V, Lewnau C J, Bullock W P, Wilson H M, Dunwell J M, Thompson J A & Wang K (1994) Production of fertile transgenic maize plants by silicon carbide whisker-mediated transformation, The Plant Journal 6: 941-948) and viral transformation techniques (e.g. see Meyer P, Heidmann I & Niedenhof 1 (1992) The use of cassaya mosaic virus as a vector system for plants, Gene 110: 213-217). Teachings on ballistic transformation are presented in following section.

Further teachings on plant transformation may be found in EP-A-0449375.

Ballistic Transformation of Plants and Plant Tissue

As indicated, techniques for producing transgenic plants are well known in the art. Typically, either whole plants, cells or protoplasts may be transformed with a suitable nucleic acid construct encoding a zinc finger molecule or target DNA (see above for examples of nucleic acid constructs). There are many methods for introducing transforming DNA constructs into cells, but not all are suitable for delivering DNA to plant cells. Suitable methods include Agrobacterium infection (see, among others, Turpen et al., 1993, J. Virol. Methods, 42: 227-239) or direct delivery of DNA such as, for example, by PEG-mediated transformation, by electroporation or by acceleration of DNA coated particles. Acceleration methods are generally preferred and include, for example, microprojectile bombardment.

Originally developed to produce stable transformants of plant species which were recalcitrant to transformation by Agrobacterium tumefaciens, ballistic transformation of plant tissue, which introduces DNA into cells on the surface of metal particles, has found utility in testing the performance of genetic constructs during transient expression. In this way, gene expression can be studied in transiently transformed cells, without stable integration of the gene in interest, and thereby without time-consuming generation of stable transformants.

In more detail, the ballistic transformation technique (otherwise known as the particle bombardment technique) was first described by Klein et al. [1987], Sanford et al. [1987] and Klein et al. [1988] and has become widespread due to easy handling and the lack of pre-treatment of the cells or tissue in interest.

The principle of the particle bombardment technique is direct delivery of DNA-coated micro-projectiles into intact plant cells by a driving force (e.g. electrical discharge or compressed air). The micro-projectiles penetrate the cell wall and membrane, with only minor damage, and the transformed cells then express the promoter constructs.

One particle bombardment technique that can be performed uses the Particle Inflow Gun (PIG), which was developed and described by Finer et al. [1992] and Vain et al. [1993]. The PIG accelerates the micro-projectiles in a stream of flowing helium, through a partial vacuum, into the plant cells.

One of advantages of the PIG is that the acceleration of the micro-projectiles can be controlled by a timer-relay solenoid and by regulation the provided helium pressure.

The use of pressurised helium as a driving force has the advantage of being inert, leaves no residues and gives reproducible acceleration. The vacuum reduces the drag on the particles and lessens tissue damage by dispersion of the helium gas prior to impact [Finer et al. 1992].

In some cases, the effectiveness and ease of the PIG system makes it a good choice for the generation of transient transformed guar tissue, which were tested for transient expression of promoter/reporter gene fusions.

A typical protocol for producing transgenic plants (in particular moncotyledons), taken from U.S. Pat. No. 5,874,265, is described below.

An example of a method for delivering transforming DNA segments to plant cells is microprojectile bombardment. In this method, non-biological particles may be coated with nucleic acids and delivered into cells by a propelling force. Exemplary particles include those comprised of tungsten, gold, platinum, and the like.

A particular advantage of microprojectile bombardment, in addition to it being an effective means of reproducibly stably transforming both dicotyledons and monocotyledons, is that neither the isolation of protoplasts nor the susceptibility to Agrobacterium infection is required. An illustrative embodiment of a method for delivering DNA into plant cells by acceleration is a Biolistics Particle Delivery System, which can be used to propel particles coated with DNA through a screen, such as a stainless steel or Nytex screen, onto a filter surface covered with plant cells cultured in suspension. The screen disperses the tungsten-DNA particles so that they are not delivered to the recipient cells in large aggregates. It is believed that without a screen intervening between the projectile apparatus and the cells to be bombarded, the projectiles aggregate and may be too large for attaining a high frequency of transformation. This may be due to damage inflicted on the recipient cells by projectiles that are too large.

For the bombardment, cells in suspension are preferably concentrated on filters. Filters containing the cells to be bombarded are positioned at an appropriate distance below the macroprojectile stopping plate. If desired, one or more screens are also positioned between the gun and the cells to be bombarded. Through the use of techniques set forth herein one may obtain up to 1000 or more clusters of cells transiently expressing a marker gene (“foci”) on the bombarded filter. The number of cells in a focus which express the exogenous gene product 48 hours post-bombardment often range from 1 to 10 and average 2 to 3.

After effecting delivery of exogenous DNA to recipient cells by any of the methods discussed above, a preferred step is to identify the transformed cells for further culturing and plant regeneration. This step may include assaying cultures directly for a screenable trait or by exposing the bombarded cultures to a selective agent or agents.

An example of a screenable marker trait is the red pigment produced under the control of the R-locus in maize. This pigment may be detected by culturing cells on a solid support containing nutrient media capable of supporting growth at this stage, incubating the cells at, e.g., 18° C. and greater than 180 μE m⁻²s⁻¹, and selecting cells from colonies (visible aggregates of cells) that are pigmented. These cells may be cultured further, either in suspension or on solid media.

An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the bar-bialaphos selective system, bombarded cells on filters are resuspended in nonselective liquid medium, cultured (e.g. for one to two weeks) and transferred to filters overlaying solid medium containing from 1-3 mg/l bialaphos. While ranges of 1-3 mg/l will typically be preferred, it is proposed that ranges of 0.1-50 mg/l will find utility in the practice of the invention. The type of filter for use in bombardment is not believed to be particularly crucial, and can comprise any solid, porous, inert support.

Cells that survive the exposure to the selective agent may be cultured in media that supports regeneration of plants. Tissue is maintained on a basic media with hormones for about 2-4 weeks, then transferred to media with no hormones. After 2-4 weeks, shoot development will signal the time to transfer to another media.

Regeneration typically requires a progression of media whose composition has been modified to provide the appropriate nutrients and hormonal signals during sequential developmental stages from the transformed callus to the more mature plant. Developing plantlets are transferred to soil, and hardened, e.g., in an environmentally controlled chamber at about 85% relative humidity, 600 ppm CO₂, and 250 μEm⁻² s⁻¹ of light. Plants are preferably matured either in a growth chamber or greenhouse. Regeneration will typically take about 3-12 weeks. During regeneration, cells are grown on solid media in tissue culture vessels. An illustrative embodiment of such a vessel is a petri dish. Regenerating plants are preferably grown at about 19° C. to 28° C. After the regenerating plants have reached the stage of shoot and root development, they may be transferred to a greenhouse for further growth and testing.

Genomic DNA may be isolated from callus cell lines and plants to determine the presence of the exogenous gene through the use of techniques well known to those skilled in the art such as PCR and/or Southern blotting.

Several techniques exist for inserting the genetic information, the two main principles being direct introduction of the genetic information and introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

Culturing and Production

Host cells transformed with the nucleotide sequence may be cultured under conditions conducive to the production of the encoded enzyme and which facilitate recovery of the enzyme from the cells and/or culture medium.

The medium used to cultivate the cells may be any conventional medium suitable for growing the host cell in questions and obtaining expression of the enzyme. Suitable media are available from commercial suppliers or may be prepared according to published recipes (e.g. as described in catalogues of the American Type Culture Collection).

The protein produced by a recombinant cell may be displayed on the surface of the cell. If desired, and as will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain which will facilitate purification of soluble proteins (Kroll D J et al (1993) DNA Cell Biol 12:441-53).

The enzyme may be secreted from the host cells and may conveniently be recovered from the culture medium by well-known procedures, including separating the cells from the medium by centrifugation or filtration, and precipitating proteinaceous components of the medium by means of a salt such as ammonium sulphate, followed by the use of chromatographic procedures such as ion exchange chromatography, affinity chromatography, or the like.

Secretion

Often, it is desirable for the enzyme to be secreted from the expression host into the culture medium from where the enzyme may be more easily recovered. According to the present invention, the secretion leader sequence may be selected on the basis of the desired expression host. Hybrid signal sequences may also be used with the context of the present invention.

Typical examples of heterologous secretion leader sequences are those originating from the fungal amyloglucosidase (AG) gene (glaA—both 18 and 24 amino acid versions e.g. from Aspergillus), the a-factor gene (yeasts e.g. Saccharomyces, Kluyveromyces and Hansenula) or the α-amylase gene (Bacillus).

Detection

A variety of protocols for detecting and measuring the expression of the amino acid sequence are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on the POI may be used or a competitive binding assay may be employed. These and other assays are described, among other places, in Hampton R et al (1990, Serological Methods, A Laboratory Manual, APS Press, St Paul Minn.) and Maddox D E et al (1983, J Exp Med 15 8:121 1).

A wide variety of labels and conjugation techniques are known by those skilled in the art and can be used in various nucleic and amino acid assays. Means for producing labelled hybridization or PCR probes for detecting the amino acid sequence include oligolabelling, nick translation, end-labelling or PCR amplification using a labelled nucleotide. Alternatively, the NOI, or any portion of it, may be cloned into a vector for the production of an mRNA probe. Such vectors are known in the art, are commercially available, and may be used to synthesize RNA probes in vitro by addition of an appropriate RNA polymerase such as T7, T3 or SP6 and labeled nucleotides.

A number of companies such as Pharmacia Biotech (Piscataway, N.J.), Promega (Madison, Wis.), and US Biochemical Corp (Cleveland, Ohio) supply commercial kits and protocols for these procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241. Also, recombinant immunoglobulins may be produced as shown in U.S. Pat. No. 4,816,567.

Additional methods to quantitate the expression of the amino acid sequence include radiolabeling (Melby P C et al 1993 J Immunol Methods 159:235-44) or biotinylating (Duplaa C et al 1993 Anal Biochem 229-36) nucleotides, coamplification of a control nucleic acid, and standard curves onto which the experimental results are interpolated. Quantitation of multiple samples may be speeded up by running the assay in an ELISA format where the oligomer of interest is presented in various dilutions and a spectrophotometric or calorimetric response gives rapid quantitation.

Although the presence/absence of marker gene expression suggests that the nucleotide sequence is also present, its presence and expression should be confirmed. For example, if the nucleotide sequence is inserted within a marker gene sequence, recombinant cells containing nucleotide sequences can be identified by the absence of marker gene function. Alternatively, a marker gene can be placed in tandem with a nucleotide sequence under the control of the promoter of the present invention or an alternative promoter (preferably the same promoter of the present invention). Expression of the marker gene in response to induction or selection usually indicates expression of the amino acid sequence as well.

Alternatively, host cells which contain the nucleotide sequence may be identified by a variety of procedures known to those of skill in the art. These procedures include, but are not limited to, DNA-DNA or DNA-RNA hybridization and protein bioassay or immunoassay techniques which include membrane-based, solution-based, or chip-based technologies for the detection and/or quantification of the nucleic acid or protein.

Fusion Proteins

The amino acid sequence of the present invention may be produced as a fusion protein, for example to aid in extraction and purification. Examples of fusion protein partners include glutathione-S-transferase (GST), 6×His, GAL4 (DNA binding and/or transcriptional activation domains) and (β-galactosidase. It may also be convenient to include a proteolytic cleavage site between the fusion protein partner and the protein sequence of interest to allow removal of fusion protein sequences. Preferably the fusion protein will not hinder the activity of the protein sequence.

The fusion protein may comprise an antigen or an antigenic determinant fused to the substance of the present invention. In this embodiment, the fusion protein may be a non-naturally occurring fusion protein comprising a substance which may act as an adjuvant in the sense of providing a generalised stimulation of the immune system. The antigen or antigenic determinant may be attached to either the amino or carboxy terminus of the substance.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

Additional POIs

The sequences of the present invention may be used in conjunction with one or more additional proteins of interest (POIs) or nucleotide sequences of interest (NOIs).

Non-limiting examples of POIs include: proteins or enzymes involved in starch metabolism, proteins or enzymes involved in glycogen metabolism, acetyl esterases, aminopeptidases, amylases, arabinases, arabinofuranosidases, carboxypeptidases, catalases, cellulases, chitinases, chymosin, cutinase, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, hemicellulases, hexose oxidases, hydrolases, invertases, isomerases, laccases, lipases, lyases, mannosidases, oxidases, oxidoreductases, pectate lyases, pectin acetyl esterases, pectin depolymerases, pectin methyl esterases, pectinolytic enzymes, peroxidases, phenoloxidases, phytases, polygalacturonases, proteases, rhamno-galacturonases, ribonucleases, thaumatin, transferases, transport proteins, transglutaminases, xylanases, hexose oxidase (D-hexose: O₂-oxidoreductase, EC 1.1.3.5) or combinations thereof. The NOI may even be an antisense sequence for any of those sequences.

The POI may even be a fusion protein, for example to aid in extraction and purification.

Examples of fusion protein partners include the maltose binding protein, glutathione-S-transferase (GST), 6×His, GAL4 (DNA binding and/or transcriptional activation domains) and β-galactosidase. It may also be convenient to include a proteolytic cleavage site between the fusion components.

The POI may even be fused to a secretion sequence. Examples of secretion leader sequences are those originating from the amyloglucosidase gene, the α-factor gene, the α-amylase gene, the lipase A gene, the xylanase A gene.

Other sequences can also facilitate secretion or increase the yield of secreted POI. Such sequences could code for chaperone proteins as for example the product of Aspergillus niger cyp B gene described in UK patent application 9821198.0.

The NOI may be engineered in order to alter their activity for a number of reasons, including but not limited to, alterations which modify the processing and/or expression of the expression product thereof. For example, mutations may be introduced using techniques which are well known in the art, e.g., site-directed mutagenesis to insert new restriction sites, to alter glycosylation patterns or to change codon preference. By way of further example, the NOI may also be modified to optimise expression in a particular host cell. Other sequence changes may be desired in order to introduce restriction enzyme recognition sites.

The NOI may include within it synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the NOI may be modified by any method available in the art. Such modifications may be carried out in to enhance the in vivo activity or life span of the NOI.

The NOI may be modified to increase intracellular stability and half-life. Possible modifications include, but are not limited to, the addition of flanking sequences of the 5′ and/or 3′ ends of the molecule or the use of phosphorothioate or 2′ O-methyl rather than phosphodiesterase linkages within the backbone of the molecule.

Antibodies

One aspect of the present invention relates to amino acid sequences that are immunologically reactive with the amino acid sequence encoded by SEQ ID No. 1.

Antibodies may be produced by standard techniques, such as by immunisation with the substance of the invention or by using a phage display library.

For the purposes of this invention, the term “antibody”, unless specified to the contrary, includes but is not limited to, polyclonal, monoclonal, chimeric, single chain, Fab fragments, fragments produced by a Fab expression library, as well as mimetics thereof. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies. Neutralising antibodies, i.e., those which inhibit biological activity of the substance polypeptides, are especially preferred for diagnostics and therapeutics.

If polyclonal antibodies are desired, a selected mammal (e.g., mouse, rabbit, goat, horse, etc.) is immunised with the sequence of the present invention (or a sequence comprising an immunological epitope thereof). Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to the sequence of the present invention (or a sequence comprising an immunological epitope thereof) contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against the sequence of the present invention (or a sequence comprising an immunological epitope thereof) can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against orbit epitopes can be screened for various properties; i.e., for isotype and epitope affinity.

Monoclonal antibodies to the sequence of the present invention (or a sequence comprising an immunological epitope thereof) may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole el al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity can be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) can be adapted to produce the substance specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse W D et al (1989) Science 256:1275-1281).

Large Scale Application

In one preferred embodiment of the present invention, the amino acid sequence is used for large scale applications.

Preferably the amino acid sequence is produced in a quantity of from 1 g per litre to about 2 g per litre of the total cell culture volume after cultivation of the host organism. Preferably the amino acid sequence is produced in a quantity of from 100 mg per litre to about 900 mg per litre of the total cell culture volume after cultivation of the host organism.

Preferably the amino acid sequence is produced in a quantity of from 250 mg per litre to about 500 mg per litre of the total cell culture volume after cultivation of the host organism.

The invention is further illustrated in the following non-limiting examples and with reference to the following figures wherein:

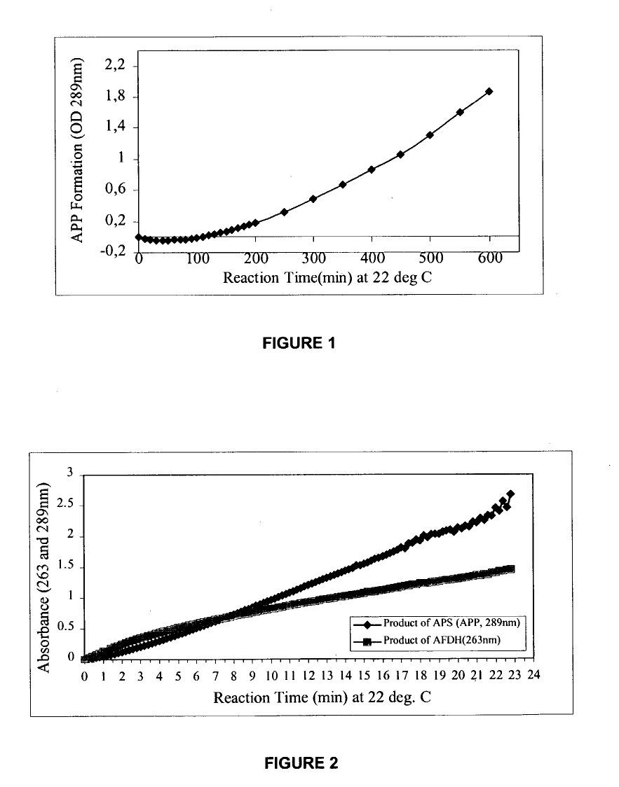

FIG. 1 shows the time course of APP formation from amylopectin as monitored at 289 nm. The reaction mixture consisted of 0.4 ml 4% (w/v) potato amylopectin, 0.16 ml 0.1 M Na-Pi buffer pH 6.5, 30 μl of enzyme mixture of glucan lyase, AFDH and APS, 0.21 ml 1 N NaCl and water to a total volume of 0.8 ml. The reaction was started by autozeroing and following the APP formation as absorbance change at 289 nm. The reaction was performed at 22° C.

FIG. 2 shows the time course of APP formation and its precursor (APM) from AF as monitored at 289 nm and 263 nm, respectively. The reaction mixture consisted of 0.4 ml 3% (w/v) AF, 0.16 ml 50 mM sodium phosphate buffer (pH 6.5), 30 pl mixture of AFDH and APS, 0.21 ml 1 N NaCl and water to a total volume of 0.8 ml. The reaction was started by autozeroing and following the APP and APM formation as absorbance changes at 289 nm and 263 nm. The reaction was performed at 22° C.

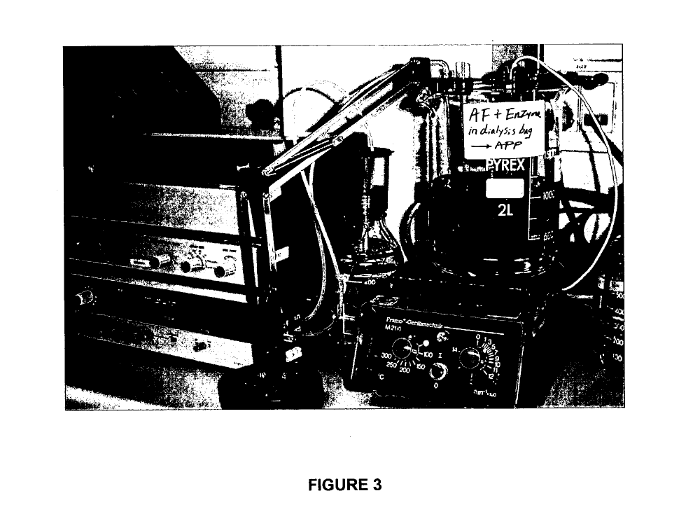

FIG. 3 shows the production of APP from AF, AFDH and APS, using 1 liter stirred membrane reactor at 24° C. under controlled pH at pH 6.2 using a pH control unit and 0.5M NaOH as the neutralizing agent. The reaction mixture consisted of 2.6% (w/v)AF, 20 mM MgCl₂ and 25 ml enzyme mixture of AFDH and APS trapped in a dialysis bag with a molecular cutoff of 8 kDa. The conversion of AF to APP was continually monitored by HPLC using a C18 column and water as the eluent. A 60% conversion of AF to APP was achieved at day 7.

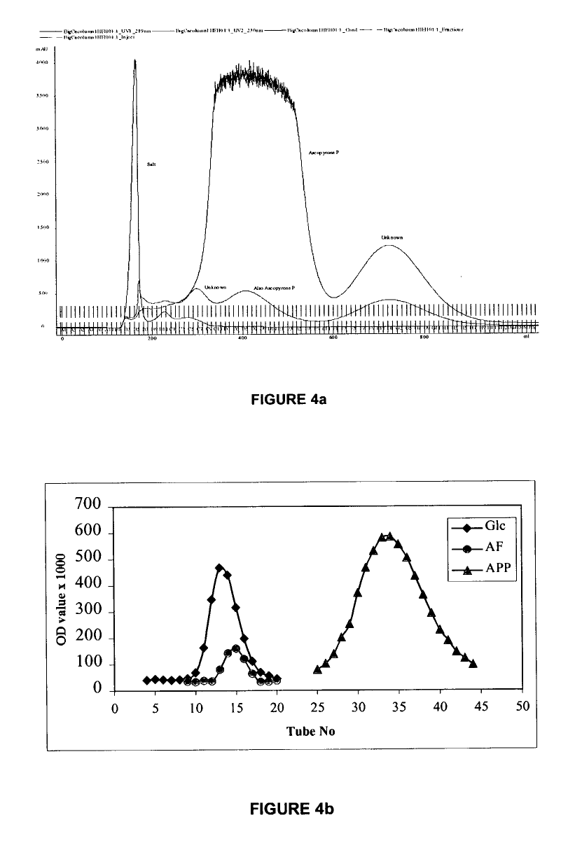

FIG. 4 shows the separation and purification of APP from AF and glucose (Glc) on a 2.6×60 cm column packed with Monosphere beads of 99Ca/320 ion cation exchanger resin (Dow Chemical Company). APP peak was detected by its max abs at 289 nm, while AF was assayed by the DNS method [Yu, S.; Olsen, CE, Marcussen, J. Methods for the assay of 1,5-anhydro-D-fructose and α-1,4-glucan lyase. Carbohydr. Res. 305: 73-82 (1998)]. Glucose was assayed by using glucose oxidase and peroxidase assay kit from Merck Co (FIG. 4b).

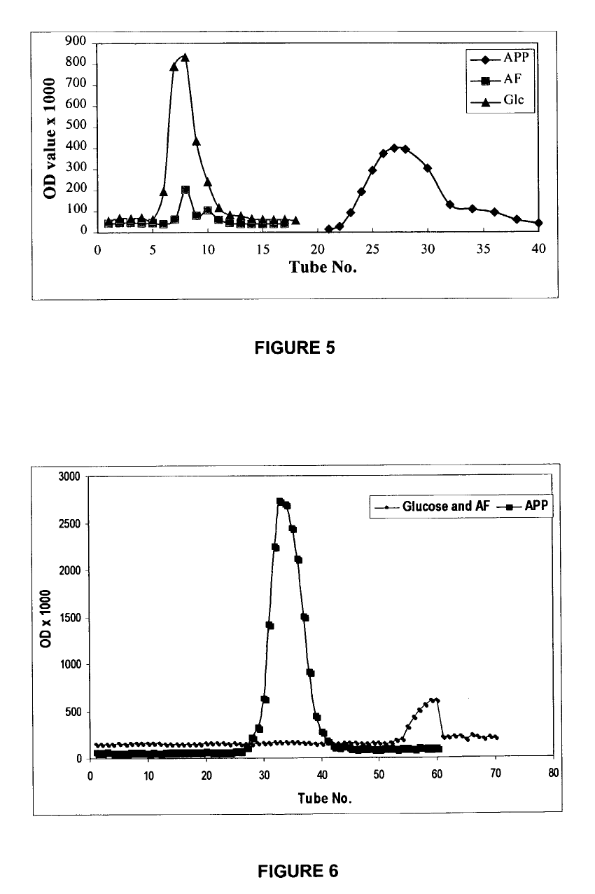

FIG. 5 shows the reverse phase separation and purification of APP from AF and glucose (Glc) on a 1.2×7.0 cm preparative C18 column from Biotage, Dyax (Charlotteville, Va.) using water as the eluent. APP, AF and Glc were analyzed as described in described in FIG. 4. FIG. 5 shows that APP eluted out at last while AF and glucose eluted out first.

FIG. 6 shows the normal phase separation and purification of APP from AF and glucose (Glc) on a 1.2×7.0 cm preparative C18 column from Biotage, Dyax (Charlotteville, Va.) using a solvent consisting 80% (v/v) acetonitrile and 20% (v/v)water. APP, AF and Glc were analyzed as described in described in FIG. 4. FIG. 6 shows that APP was eluted out first and AF and glucose eluted out last.

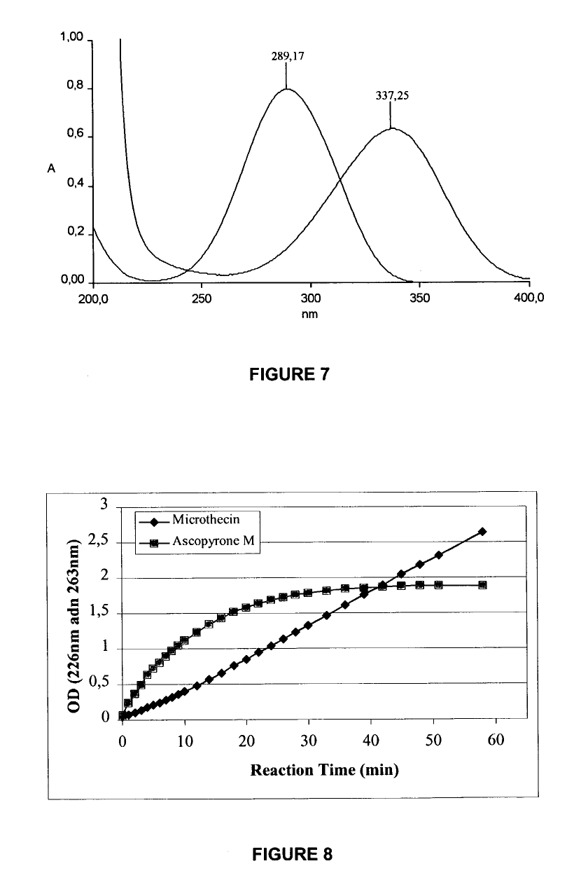

FIG. 7 shows that selectively extracted APP product exhibited typical APP absorbance peak in water at 289 nm and in 20 mM NaOH at 337 nm when scanned between 400 and 200 nm using an uv/vis spectrophotometer. The APP was prepared from 10% (w/v) dextrins using an enzyme mixture of α-1,4-glucan lyase, AFDH, and APS. The reaction was performed at pH 6.5, 22° C. for 3 days. The APP formed was extracted with 80% acetonitrile (v/v), evaporated and dissolved in water and analyzed.

FIG. 8 shows the formation of ascopyrone M and microthecin, from pyranosone dehydratase and AF, monitored at 263 and 226 nm, respectively. The reaction mixture consisted of 20 μl PD, 66 μl 3% (w/v) AF, 0.81 ml water, 0.1 ml sodium phosphate buffer (pH 6.5, 0.1M). The reaction was started at 22° C. by adding the enzyme PD.

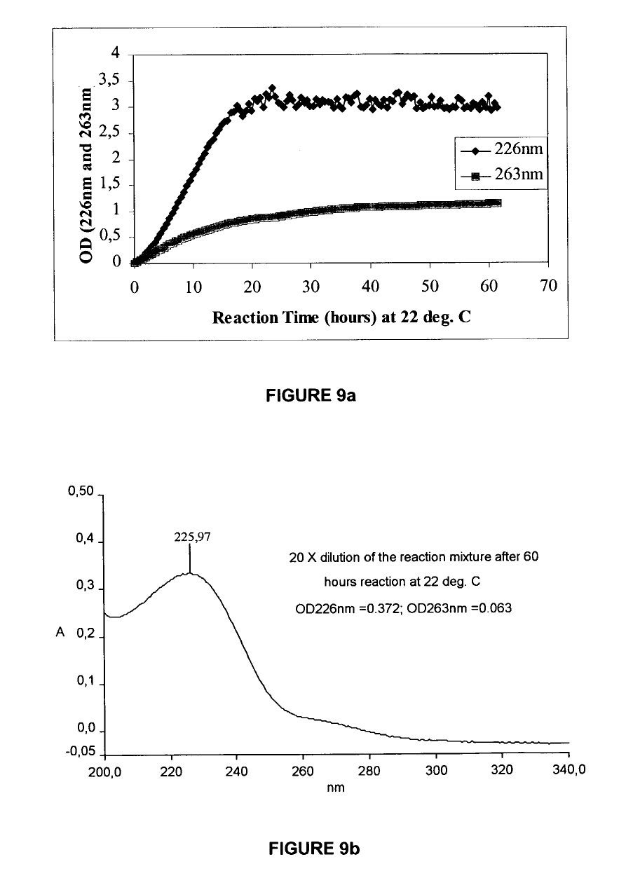

FIG. 9 shows the formation of microthecin and its intermediate (ascopyrone M) from dextrin, glucan lyase and pyranosone dehydratase. The reaction mixture consisted of 20 μl purified glucan lyase and 10 μl pyranosone dehydratase in 25 mM sodium phosphate buffer (pH 6.5) and 0.35 ml 2% (w/v) dextrin 10 (Fluka Chemie AG, Buchs, Switzerland), 70 μl sodium phosphate buffer (pH 6.5, 0.1 M) and water 0.25 ml. The formation of microthecin and ascopyrone M were monitored at 226 and 263 nm at 22° C. using an uv/vis spectrophotometer (which had a measuring range of OD 0 to 2.5) for 62 hours (FIG. 9a). At the end of the reaction, the mixture was diluted 20 times in water and scanned between 340 nm and 200 nm and the peak of microthecin detected at 226 nm (FIG. 9b).

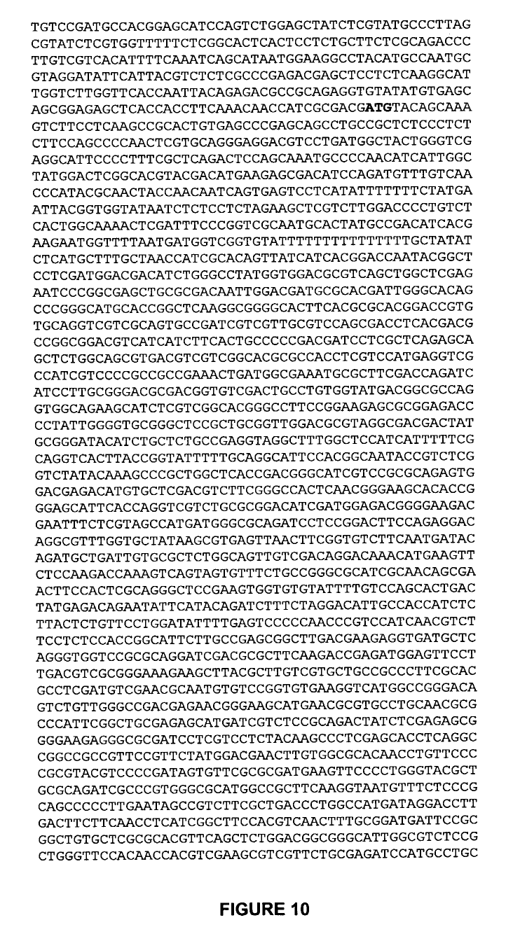

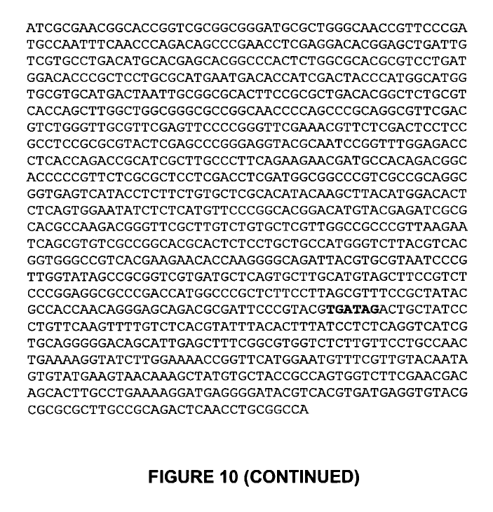

FIG. 10 (SEQ ID NO. 1) shows the gene coding for pyranosone dehydratase (PD) from the fungus Phanerochaete chrysosporium including the upstream regulatory region (−1 to −288), the coding region (1-3146) and down-stream region (3147-3444). The presumed starch coden is ATG (bold) and stop codens are TGA TAG(bold). The purified functional PD corresponds to a N-terminal 7-amino acid truncated PD if the translation is assumed to start from the bold coden ATG.

### EXAMPLES

1. Preparation of APP by Using Free Enzymes

APP was produced by one step in one pot. Starch-typed substrates, such as starch, amylopectin, amylose and dextrins are incubated with α-1,4-glucan lyase, AFDH and APS. The starch-typed substrate had a concentration of 2 to 20% (w/v). Isoamylase and pullulanase as auxiliary enzymes were added to the reaction mixture to increase the AF yield and therefore the yields of APM, APP and microthecin. The reaction was performed at 22 to 75° C.

Alternatively, Starch-typed substrates, such as starch, amylopectin, amylose and dextrins are first incubated with α-1,4-glucan lyase in the pH range form 3.8 to 7.0. The starch-typed substrate has a concentration of 2 to 35% (w/v). Isoamylase and pullulanase were added to the reaction mixture to increase the AF yield. The reaction was performed at 22 to 75° C. The produced AF is not separated from un-reacted substrate and by products (such as glucose) and a mixture of AFDH and APS is added to convert the formed AF to APP. Such further incubation lasted for 1 to 7 days at 22 to 75° C. and at pH from 5.0 to 7.5, preferably at pH 6.2 controlled by the addition of concentrated HCl and NaOH.

Alternatively, AF produced from Starch-typed substrates, such as starch, amylopectin, amylose and dextrins is separated from un-reacted substrate and by products (such as glucose) by ultrafiltration. The separated AF is used as a substrate for AFDH and APS to form APP. AF used is in a concentration of 0.4 to 20% (w/v). These reactions are performed at 22 to 45° C., preferably at 24° C. for 1 to 7 days. The pH of the reaction mixture is kept at pH 5.5 to 7.5, preferably at pH 6.2 and controlled by the addition of concentrated HCl and NaOH. Divalent salts, such as 10-20 mM CaCl₂ and/or 0.5M NaCl may be added to the reaction mixture to stabilize the enzymes and the APP formed.

The production of APP is followed and quantified spectrophotometrically by monitoring the absorbance at 289 nm and by monitoring the concentration of glucose, AF and APP on a Waters HPLC instrument (model WISP 710B) equipped with a differential refractometer (model 410) and a uv monitor (Lambda-Max model 481 LC spectrophotometer) set at 289 nm. The column used is a carbohydrate Ca²⁺ column (6.5×300 mm, Interaction Chromatography Inc. San Jose, Calif.) and a symmetry shield C18 column (3.9×150 mm, Waters Corporation). The structure of APP was confirmed using NMR as described earlier (Andersen et al., 2002).

The APP formed is also analyzed by TLC. The solvent system was composed of chloroform:Methanol=65:35. A aluminun silica gel 60 TLC plate (0.2 mm thick and with or with out fluroescen indicator) of 20×20 cm from Merck is used. The APP samples are applied on the origin in a volume of 1-2 μl and the plate is then developed upward in the above solvent system at room temperature. APP, AF, glucose and other sugars on the TLC plates were well separated and revealed by spraying a reagent of 25 ml acetic acid, 0.5 ml concentrated sulfuric acid and 0.25 ml anisaldehyde and then warmed at around 110° C. on bread toaster for 5-10 min.

2. Preparation of APP by Using Immobilized Enzymes

Immobilization of the fungal AFDH and APS in activity ratio of 1:1 was achieved by using succinimide-activated Sepharose (Affigel 15 gel, Bio-Rad Laboratories) and glutardialdehyde-activated silica affinity adsorbent (Boehringer Mannheim). The recovery of AFDH and APS activity after immobilization on Affigel 15 gel varied between 40 to 50%. The immobilized enzymes showed good stability. The operational stability of a column packed with the immobilized lyase was at least 16 days when operated at 22 to 75° C. at pH 6.0.

With AFDH and APS immobilized on glutardialdehyde-activated silica, the recovery was 80-100%.

In the above described process, AFDH could replaced with a pyranosone dehydratase (PD) from Phanerochaete chrysosporium and microthecin is produced or APP is produced if APS is present.

3. Separation and Purification of APP from AF, Glucose, Salt and Other Impurities

When starch, amylopectin or dextrins were used as substrate, the products besides APP were AF, glucose, maltosaccharides, and limit dextrins. The separation of un-reacted substrate and limit dextrins and the enzyme was achieved by ultrafiltration with membranes with molecule cutoff in the range of 300 to 100,000, preferably 3,000 and 10,000.

Alternatively, the APP was selectively extracted from the reaction mixture using an organic solvent selected from acetonitrile, ethylacetate, ethanol, propanol, isopropanol, acetone, and butanol, preferably acetonitrile in a final concentration of 80-90% (v/v). The organic solvent was removed from the APP by evaporation under reduced pressure and recycled.

APP was further separated from AF, glucose, maltose and maltodextrins by ion exchange chromatography and gel filtration. Preferably the count ion in the cation chromatography is selected among, Ca²⁺, Pb²⁺, Na¹⁺, H¹⁺, K¹⁺, and Ag¹⁺. The cation exchanger resins are selected from polymers such polyethylene, chemically modified dextrans, agarose, preferably polystyrene-divinylbenzene copolymer with sulfonic acid as the active group, for example, monosphere resin 99Ca/320 from Dow Chemical Co. Ltd. For gel filtration, the medium matrices are based on cross-linked dextrans, agarose, polyacrylaminde and polystyrene-divinylbenzene copolymer.

Alternatively the formed APP was separated from AF, glc, maltose, maltosaccharides, salts etc by reversed phase chromatography on media with the functional group of C8 or C18. Such as C18 medium from Biotage, Dyax (Charlotteville, Va.) and C18 symmetry shield separating material from Waters Corporation.

Alternatively, APP was separated from AF, glc, maltose, maltosaccharides, salts etc by normal phase chromatography on silica gel with a solvent system of or CHCl₂-ethanol or chloroform methanol (65:35) or acetonitrile-water (80:20).

AF and APP were de-ashed through a process consists of a strong acid cation followed by a weak base anion to remove all the salt and other undesired products. This cation/anion system can either be single pass or double pass (double pass=cation/anion/cation/anion). For polishing purposes, AF or APP are post-treated with a mixed bed ion exchange resin. This mixed bed consists of a strong acid cation and a type 2 strong base anion resin (maximum operating temperature 45° C.). For example, the choice of mixed bed resin for polishing are DOWEX* 88 MB (strong acid cation) and DOWEX 22 (strong base anion) from Dow Chemical Co. Ltd.

The APP was either concentrated under reduced pressure, or crystallized in organic solvent.

The produced APP was analyzed by TLC, HPLC, spectrophotometry and ¹³C-NMR.

4. Preparation of Other Anhydrofructose Derivatives

Other 1,5-anhydro-D-fructose derivatives (AFDs), for example, microthecin, can be produced in the same way as APP described above. For example, microthecin can be prepared from AF using PD from P. chrysosporium. PD can be in either free form or immobilized form as in the cases of AFDH and APS. The reaction conditions were the same as for APP described above.

All publications mentioned in the above specification are herein incorporated by reference. Various modifications and variations of the described methods and systems of the invention will be apparent to those skilled in the art without departing from the scope and spirit of the invention. Although the invention has been described in connection with specific preferred embodiments, it should be understood that the invention as paragraphed should not be unduly limited to such specific embodiments. Indeed, various modifications of the described modes of carrying out the invention which are obvious to those skilled in molecular biology or related fields are intended to be within the scope of the following paragraphs.

The invention will now be further described by the following numbered paragraphs:

1. A process for preparing ascopyrone P, or a derivative thereof, said process comprising the steps of:

(I) converting a starch-type substrate to 1,5-anhydro-D-fructose with α-1,4-glucan lyase at a pH of from about 3.8 to 7.0;

(II) treating said 1,5-anhydro-D-fructose with 1,5-anhydro-D-fructose dehydratase and/or pyranosone dehydratase and optionally ascopyrone P synthase at a pH of from about 5.0 to about 7.5.

2. A process according to any preceding paragraph wherein steps (I) and (II) are carried out in a one-pot process by forming a reaction mixture comprising a starch-type substrate, α-1,4-glucan lyase, 1,5-anhydro-D-fructose dehydratase and/or pyranosone dehydratase, and optionally ascopyrone P synthase wherein the process is carried out at a pH of from about 5.0 to 7.5.

3. A process according to paragraph 2 which is carried out at a pH of from about 5.0 to about 7.0.

4. A process according to paragraph 2 or paragraph 3 wherein the concentration of starch-type substrate is from 2 to 20% (w/v).

5. A process according to paragraph I wherein steps (I) and (II) are carried out sequentially.

6. A process according to paragraph 5 comprising:

(a) forming a reaction mixture comprising a starch-type substrate and α-1,4-glucan lyase; and

(b) adding 1,5-anhydro-D-fructose dehydratase and/or pyranosone dehydratase and optionally ascopyrone P synthase thereto.

7. A process according to any preceding paragraph which is carried out at a temperature from about 22° C. to about 75° C.

8. A process according to any one of paragraphs 5 to 7 wherein the concentration of starch-type substrate is from about 2 to about 35% (w/v).

9. A process according to paragraph 5 comprising:

(a) forming a first reaction mixture comprising a starch-type substrate and α-1,4-glucan lyase;

(b) isolating 1,5-anhydro-D-fructose obtained from said first reaction mixture;

(c) forming a second reaction mixture comprising 1,5-anhydro-D-fructose, 1,5-anhydro-D-fructose dehydratase and/or pyranosone dehydratase and optionally ascopyrone P synthase.

10. A process according to paragraph 9 wherein the 1,5-anhydro-D-fructose is isolated from said first reaction mixture by ultrafiltration.

11. A process according to paragraph 9 or paragraph 10 wherein the concentration of 1,5-anhydro-D-fructose in said second reaction mixture is from about 0.4 to about 20% (w/v).

12. A process according to any one of paragraphs 9 to 11 which is carried out at a temperature of from about 22° C. to about 45° C.

13. A process for preparing ascopyrone P in accordance with paragraph 1, said process comprising the steps of:

(I) converting a starch-type substrate to 1,5-anhydro-D-fructose with α-1,4-glucan lyase at a pH of from about 3.8 to 7.0;

(II) treating said 1,5-anhydro-D-fructose with 1,5-anhydro-D-fructose dehydratase or pyranosone dehydratase, and ascopyrone P synthase at a pH of from about 5.0 to about 7.5.

14. A process according to paragraph 13 wherein steps (I) and (II) are carried out in a one-pot process by forming a reaction mixture comprising a starch-type substrate, α-1,4-glucan lyase, 1,5-anhydro-D-fructose dehydratase or pyranosone dehydratase, and ascopyrone P synthase, wherein said process is carried out at a pH of from about 5.0 to about 7.5.

15. A process according to any preceding paragraph wherein said derivative of ascopyrone P is microthecin or ascopyrone M.

16. A process for preparing microthecin in accordance with paragraph 1, said process comprising the steps of:

(I) converting a starch-type substrate to 1,5-anhydro-D-fructose with α-1,4-glucan lyase at a pH of from about 3.8 to 7.0;

(II) converting said 1,5-anhydro-D-fructose to microthecin with pyranosone dehydratase and optionally 1,5-anhydro-D-fructose dehydratase at a pH of from about 5.0 to about 7.5.

17. A process preceding paragraph 16 wherein steps (I) and (II) are carried out in a one-pot process by forming a reaction mixture comprising a starch-type substrate, α-1,4-glucan lyase, pyranosone dehydratase and optionally 1,5-anhydro-D-fructose dehydratase, wherein the process is carried out at a pH of from about 5.0 to about 7.5.

18. A process for preparing ascopyrone M in accordance with paragraph 1, said process comprising the steps of:

(I) converting a starch-type substrate to 1,5-anhydro-D-fructose with α-1,4-glucan lyase at a pH of from about 3.8 to 7.0;

(II) converting said 1,5-anhydro-D-fructose to ascopyrone M with pyranosone dehydratase or 1,5-anhydro-D-fructose dehydratase at a pH of from about 5.0 to about 7.5.

19. A process according to paragraph 18 wherein steps (I) and (II) are carried out in a one-pot process by forming a reaction mixture comprising a starch-type substrate, α-1,4-glucan lyase, and pyranosone dehydratase or 1,5-anhydro-D-fructose dehydratase, wherein the process is carried out at a pH of from about 5.0 to about 7.5.

20. A process according to paragraph 2, 14, 17 or 19 wherein the pH is between about 6.0 and 6.5.

21. A process according to any preceding paragraph wherein the starch-type substrate is selected from starch, amylopectin, maltosaccharides, amylose and dextrin.

22. A process according to any preceding paragraph which further comprises the use of isoamylase and/or pullalanase.

23. A process according to any preceding paragraph which further comprises the use of one or more divalent metal salts.

24. A process according to paragraph 23 wherein said divalent metal salt is selected from NaCl and CaCl₂.

25. A process according to any preceding paragraph wherein the reaction time is from 1 to 7 days.

26. A process according to any preceding paragraph wherein said α-1,4-glucan lyase and/or 1,5-anhydro-D-fructose dehydratase and/or pyranosone dehydratase and/or ascopyrone P synthase are in free form.

27. A process according to any preceding paragraph wherein said α-1,4-glucan lyase and/or 1,5-anhydro-D-fructose dehydratase and/or pyranosone dehydratase and/or ascopyrone P synthase are immobilised on a support.

28. A process according to paragraph 27 said wherein said α-1,4-glucan lyase and/or 1,5-anhydro-D-fructose dehydratase and/or pyranosone dehydratase and/or ascopyrone P synthase are immobilised on a succinimide-activated or a glutardiadehyde-activated solid support.

29. A process according to any one of paragraphs 1 to 25 said wherein said α-1,4-glucan lyase and/or 1,5-anhydro-D-fructose dehydratase and/or pyranosone dehydratase and/or ascopyrone P synthase are held in membrane containers.

30. A process according to any preceding paragraph wherein said ascopyrone P or derivative thereof is purified by selective extraction.

31. A process according to paragraph 30 wherein said ascopyrone P or derivative thereof is extracted with an organic solvent selected from acetonitrile, ethyl acetate, ethanol, propanol, isopropanol, acetone and butanol.

32. A process according to any preceding paragraph wherein said ascopyrone P or derivative thereof is concentrated under reduced pressure and optionally crystallised from an organic solvent.

33. A process according to any preceding paragraph wherein said ascopyrone P or derivative thereof is purified by reverse phase or normal phase chromatography.

34. A process according to any one of paragraphs 1 to 32 wherein said ascopyrone P or derivative thereof is purified by ion exchange chromatography and/or gel filtration.

35. A process according to any preceding paragraph wherein said ascopyrone P or derivative thereof is freeze dried or spray dried.

36. A process according to paragraph 1 wherein step (II) comprises converting said 1,5-anhydro-D-fructose to ascopyrone P with ascopyrone P synthase and 1,5-anhydro-D-fructose dehydratase.

37. A process according to paragraph 36 wherein said 1,5-anhydro-D-fructose dehydratase is characterised by one or more of the following:

(a) having a temperature optimum of from about 34 to 50° C.;

(b) having an optimal pH range of from about 5.9 to about 7.0;

(c) being stable in 50 mM sodium phosphate buffer (pH 7.0) containing 0.1 M NaCl for at least two weeks at 4° C.; or

(d) exhibiting enhanced activity in the presence of Mg²⁺, Ca²⁺ or Na²⁺ ions;

(e) being inhibited in the presence of ZnCl₂, EDTA or DTT.

38. A process according to paragraph 1 wherein step (II) comprises converting said 1,5-anhydro-D-fructose to ascopyrone P with ascopyrone P synthase and pyranosone dehydratase.

39. A process according to paragraph 38 wherein said pyranosone dehydratase is encoded by the nucleotide sequence set forth in SEQ. ID. No.1.

40. A process according to paragraph 38 wherein said pyranosone dehydratase comprises at least one sequence selected from the following:

| | |
|---|---|
| (i) | KPHCEPEQPAALPLFQPQLVQGGRPDXYWVEAFPFRSDSSK or |
| | KPHXEPEQPAALPLFQPQLVV(Q)GGRPDXY; |
| | KPHXEPEQPAALPLFQPQLVV(Q)GGRPDXY |
| (ii) | SDIQMFVNPYATTNNQSSXWTPVSLAKLDFPVAMHYADITK; |
| (iii) | VSWLENPGELR; |
| (iv) | DGVDCLWYDGAR; |
| (v) | PAGSPTGIVRAEWTRHVLDVFGXLXXK; |
| (vi) | HTGSIHQVVCADIDGDGEDEFLVAMMGADPPDFQRTGVWCYK; |
| (vii) | TEMEFLDVAGK; |
| (viii) | KLTLVVLPPFARLDVERNVSGVK; |
| (ix) | SMDELVAHNLFPAYVPDSVR; |
| (x) | NDATDGTPVLALLDLDGGPSPQAWNISHVPPGTDMYEIAHAK; |
| (xi) | TGSLVCARWPPVK; |
| (xii) | NQRVAGTHSPAAMGLTSRWAVTK; |
| (xiii) | GQITFRLPEAPDHGPLFLSVSAIRHQ; |

or a variant, homologue, fragment or derivative thereof.

41. A process according to paragraph 38 wherein said ascopyrone P synthase is characterised by one or more of the following:

(a) having an optimim temperature range of 25 to 50° C.;

(b) having an optimal pH range of from about 4.5 to 7.5;

(c) being stable in 50 mM sodium phosphate buffer (pH 7.0) containing 0.1 M NaCl for at least one month at 4° C.; or

(d) comprising at least one amino acid sequence selected from (i) AINLPFSNWAX(or C)TI and (ii) EYGRTFFTRYDYENVD.

42. A process according to any preceding paragraph wherein said α-1,4-glucan lyase, ascopyrone P synthase, 1,5-anhydro-D-fructose dehydratase and pyranosone dehydratase have a purity of greater than 90%.

43. A process according to any preceding paragraph wherein said α-1,4-glucan lyase, ascopyrone P synthase, 1,5-anhydro-D-fructose dehydratase and pyranosone dehydratase are in pure or substantially pure form.

44. A process for preparing ascopyrone P, microthecin or ascopyrone M substantially as described herein and with reference to the accompanying Examples.

1. A process for preparing ascopyrone P, or a derivative thereof, said process comprising the steps of:
(i) converting a starch-type substrate to 1,5-anhydro-D-fructose with α-1,4-glucan lyase at a pH of from about 3.8 to 7.0;
(ii) treating said 1,5-anhydro-D-fructose with 1,5-anhydro-D-fructose dehydratase and/or pyranosone dehydratase and optionally ascopyrone P synthase at a pH of from about 5.0 to about 7.5.

2. The process according to claim 1 wherein steps (I) and (II) are carried out in a one-pot process by forming a reaction mixture comprising a starch-type substrate, α-1,4-glucan lyase, 1,5-anhydro-D-fructose dehydratase and/or pyranosone dehydratase, and optionally ascopyrone P synthase wherein the process is carried out at a pH of from about 5.0 to 7.5.

3. The process according to claim 2 which is carried out at a pH of from about 5.0 to about 7.0.

4. The process according to claim 2 wherein the concentration of starch-type substrate is from 2 to 20% (w/v).

5. The process according to claim 1 wherein steps (I) and (II) are carried out sequentially.

6. The process according to claim 5 comprising:
(a) forming a reaction mixture comprising a starch-type substrate and α-1,4-glucan lyase; and
(b) adding 1,5-anhydro-D-fructose dehydratase and/or pyranosone dehydratase and optionally ascopyrone P synthase thereto.

7. The process according to claim 1 which is carried out at a temperature from about 22° C. to about 75° C.

8. The process according to claim 5 wherein the concentration of starch-type substrate is from about 2 to about 35% (w/v).

9. The process according to claim 5 comprising:
(a) forming a first reaction mixture comprising a starch-type substrate and α-1,4-glucan lyase;
(b) isolating 1,5-anhydro-D-fructose obtained from said first reaction mixture;
(c) forming a second reaction mixture comprising 1,5-anhydro-D-fructose, 1,5-anhydro-D-fructose dehydratase and/or pyranosone dehydratase and optionally ascopyrone P synthase.

10. The process according to claim 9 wherein the 1,5-anhydro-D-fructose is isolated from said first reaction mixture by ultrafiltration.

11. The process according to claim 9 wherein the concentration of 1,5-anhydro-D-fructose in said second reaction mixture is from about 0.4 to about 20% (w/v).

13. The process according to claim 9 which is carried out at a temperature of from about 22° C. to about 45° C.

13. A process for preparing ascopyrone P in accordance with claim 1, said process comprising the steps of:
(i) converting a starch-type substrate to 1,5-anhydro-D-fructose with α-1,4-glucan lyase at a pH of from about 3.8 to 7.0;
(ii) treating said 1,5-anhydro-D-fructose with 1,5-anhydro-D-fructose dehydratase or pyranosone dehydratase, and ascopyrone P synthase at a pH of from about 5.0 to about 7.5.

14. The process according to claim 13 wherein steps (i) and (ii) are carried out in a one-pot process by forming a reaction mixture comprising a starch-type substrate, α-1,4-glucan lyase, 1,5-anhydro-D-fructose dehydratase or pyranosone dehydratase, and ascopyrone P synthase, wherein said process is carried out at a pH of from about 5.0 to about 7.5.

15. The process according to claim 1 wherein said derivative of ascopyrone P is microthecin or ascopyrone M.

17. A process for preparing microthecin in accordance with claim 1, said process comprising the steps of:
(i) converting a starch-type substrate to 1,5-anhydro-D-fructose with α-1,4-glucan lyase at a pH of from about 3.8 to 7.0;
(ii) converting said 1,5-anhydro-D-fructose to microthecin with pyranosone dehydratase and optionally 1,5-anhydro-D-fructose dehydratase at a pH of from about 5.0 to about 7.5.

17. The process according to claim 16 wherein steps (i) and (ii) are carried out in a one-pot process by forming a reaction mixture comprising a starch-type substrate, α-1,4-glucan lyase, pyranosone dehydratase and optionally 1,5-anhydro-D-fructose dehydratase, wherein the process is carried out at a pH of from about 5.0 to about 7.5.

18. A process for preparing ascopyrone M in accordance with claim 1, said process comprising the steps of:
(i) converting a starch-type substrate to 1,5-anhydro-D-fructose with α-1,4-glucan lyase at a pH of from about 3.8 to 7.0;
(ii) converting said 1,5-anhydro-D-fructose to ascopyrone M with pyranosone dehydratase or 1,5-anhydro-D-fructose dehydratase at a pH of from about 5.0 to about 7.5.

19. The process according to claim 18 wherein steps (i) and (ii) are carried out in a one-pot process by forming a reaction mixture comprising a starch-type substrate, α-1,4-glucan lyase, and pyranosone dehydratase or 1,5-anhydro-D-fructose dehydratase, wherein the process is carried out at a pH of from about 5.0 to about 7.5.

20. The process according to claim 2 wherein the pH is between about 6.0 and 6.5.

22. The process according to claim 1 wherein the starch-type substrate is selected from starch, amylopectin, maltosaccharides, amylose and dextrin.

22. The process according to claim 1 which further comprises the use of isoamylase and/or pullalanase.

23. The process according to claim 1 which further comprises the use of one or more divalent metal salts.

24. The process according to claim 23 wherein said divalent metal salt is selected from the group consisting of NaCl and CaCl₂.

26. The process according to claim 1 wherein the reaction time is from 1 to 7 days.

26. The process according to claim 1 wherein said α-1,4-glucan lyase and/or 1,5-anhydro-D-fructose dehydratase and/or pyranosone dehydratase and/or ascopyrone P synthase are in free form.

27. The process according to claim 1 wherein said α-1,4-glucan lyase and/or 1,5-anhydro-D-fructose dehydratase and/or pyranosone dehydratase and/or ascopyrone P synthase are immobilised on a support.

30. The process according to claim 27 said wherein said α-1,4-glucan lyase and/or 1,5-anhydro-D-fructose dehydratase and/or pyranosone dehydratase and/or ascopyrone P synthase are immobilised on a succinimide-activated or a glutardiadehyde-activated solid support.

31. A process according to claims 1 wherein said α-1,4-glucan lyase and/or 1,5-anhydro-D-fructose dehydratase and/or pyranosone dehydratase and/or ascopyrone P synthase are held in membrane containers.

30. The process according to claim 1 wherein said ascopyrone P or derivative thereof is purified by selective extraction.

32. The process according to claim 30 wherein said ascopyrone P or derivative thereof is extracted with an organic solvent selected from acetonitrile, ethyl acetate, ethanol, propanol, isopropanol, acetone and butanol.

32. The process according to claim 1 wherein said ascopyrone P or derivative thereof is concentrated under reduced pressure and optionally crystallised from an organic solvent.

33. The process according to claim 1 wherein said ascopyrone P or derivative thereof is purified by reverse phase or normal phase chromatography.

34. The process according to claims 1 wherein said ascopyrone P or derivative thereof is purified by ion exchange chromatography and/or gel filtration.

38. The process according to claim 1 wherein said ascopyrone P or derivative thereof is freeze dried or spray dried.

39. The process according to claim 1 wherein step (ii) comprises converting said 1,5-anhydro-D-fructose to ascopyrone P with ascopyrone P synthase and 1,5-anhydro-D-fructose dehydratase.

40. The process according to claim 36 wherein said 1,5-anhydro-D-fructose dehydratase is characterised by one or more of the following:
(a) having a temperature optimum of from about 34 to 50° C.;
(b) having an optimal pH range of from about 5.9 to about 7.0;
(c) being stable in 50 mM sodium phosphate buffer (pH 7.0) containing 0.1 M NaCl for at least two weeks at 4° C.; or
(d) exhibiting enhanced activity in the presence of Mg²⁺, Ca²⁺ or Na²⁺ ions;
(e) being inhibited in the presence of ZnCl₂, EDTA or DTT.

38. The process according to claim 1 wherein step (ii) comprises converting said 1,5-anhydro-D-fructose to ascopyrone P with ascopyrone P synthase and pyranosone dehydratase.

39. The process according to claim 38 wherein said pyranosone dehydratase is encoded by the nucleotide sequence set forth in SEQ. ID. No.1.

40. The process according to claim 38 wherein said pyranosone dehydratase comprises at least one sequence selected from the following:

| | |
|---|---|
| (i) | KPHCEPEQPAALPLFQPQLVQGGRPDXYWVEAFPFRSDSSK or |
| | KPHXEPEQPAALPLFQPQLVV(Q)GGRPDXY; |
| (ii) | SDIQMFVNPYATTNNQSSXWTPVSLAKLDFPVAMHYADITK; |
| (iii) | VSWLENPGELR; |
| (iv) | DGVDCLWYDGAR; |
| (v) | PAGSPTGIVRAEWTRHVLDVFGXLXXK; |
| (vi) | HTGSIHQVVCADIDGDGEDEFLVAMMGADPPDFQRTGVWCYK; |
| (vii) | TEMEFLDVAGK; |
| (viii) | KLTLVVLPPFARLDVERNVSGVK; |
| (ix) | SMDELVAHNLFPAYVPDSVR; |
| (x) | NDATDGTPVLALLDLDGGPSPQAWNISHVPPGTDMYEIAHAK; |
| (xi) | TGSLVCARWPPVK; |
| (xii) | NQRVAGTHSPAAMGLTSRWAVTK; and |
| (xiii) | GQITFRLPEAPDHGPLFLSVSAIRHQ; |

or a variant, homologue, fragment or derivative thereof.

41. The process according to claim 38 wherein said ascopyrone P synthase is characterised by one or more of the following:
(a) having an optimim temperature range of 25 to 50° C.;
(b) having an optimal pH range of from about 4.5 to 7.5;
(c) being stable in 50 mM sodium phosphate buffer (pH 7.0) containing 0.1 M NaCl for at least one month at 4° C.; or
(d) comprising at least one amino acid sequence selected from
(i) AINLPFSNWAX(or C)TI and
(ii) (ii) EYGRTFFTRYDYENVD.

42. The process according to claim 1 wherein said α-1,4-glucan lyase, ascopyrone P synthase, 1,5-anhydro-D-fructose dehydratase and pyranosone dehydratase have a purity of greater than 90%.

43. The process according to claim 1 wherein said α-1,4-glucan lyase, ascopyrone P synthase, 1,5-anhydro-D-fructose dehydratase and pyranosone dehydratase are in pure or substantially pure form.
